(19) United States
(12) Patent Application Publication
(10) Pub. No.: US 2022/0170018 A1
(43) Pub. Date: Jun. 2, 2022

(54) CONTROL OF INSECT PESTS USING RNA MOLECULES

(71) Applicant: DEVGEN NV, Gent - Zwijnaarde (BE)

(72) Inventors: Lien DE SCHRIJVER, GENT- ZWIJNAARDE (BE); Kaat CAPPELLE, GENT-ZWIJNAARDE (BE); Lies DEGRAVE, GENT-ZWIJNAARDE (BE); Myriam BEGHYN, GENT-ZWIJNAARDE (BE); Yann NAUDET, GENT-ZWIJNAARDE (BE)

(73) Assignee: DEVGEN NV, GENT- ZWIJNAARDE (BE)

(21) Appl. No.: 17/593,594

(22) PCT Filed: Mar. 13, 2021
(86) PCT No.: PCT/EP2020/056983

§ 371 Date: Sep. 21, 2021

(30) Foreign Application Priority Data
Mar. 21, 2019 (EP) 19164378.2

(51) Int. Cl. C12N 15/113; A01N 63/60; C12N 15/82; A01N 63/50

(52) U.S. Cl. CPC C12N 15/113; A01N 63/60; C12N 2310/14; A01N 63/50; C12N 15/8286; C12N 15/8218

(57) ABSTRACT

Disclosed are double stranded RNA molecules that are toxic to flea beetles, particularly the flea beetle species Psylliodes chrysocephala, Phyllotreta nemorum, Phyllotreta striolata, Phyllotreta armoraciae, Phyllotreta atra and Phyllotreta cruciferae. In particular, interfering RNA molecules capable of interfering with pest target genes and that are toxic to the target pest are provided. Further, methods of making and using the interfering RNA, for example in transgenic plants or as the active ingredient in a composition, to confer protection from insect damage are disclosed.

The invention relates generally to the control of pests that cause damage to crop plants by their feeding activities, and more particularly to the control of beetles by compositions comprising interfering RNA molecules. The invention further relates to the compositions and to methods of using such compositions comprising the interfering RNA molecules.

### BACKGROUND

Flea beetles are members of the leaf beetle family Chrysomelidae, sub family Galerucinae, in the tribe Alticini. Adult flea beetles feed externally on plants, eating the surface of leaves, stems, and petals. The flea beetles Psylliodes chrysocephala, Phyllotreta cruciferae, Phyllotreta nemorum, and Phyllotreta striolata (Coleoptera: Chrysomelidae) are significant pests of crops in the Brassicaceae family, including Brussel sprouts, kale, broccoli, cauliflower, and canola (Brassica rapa and Brassica napus). Flea beetles are primarily controlled by application of chemical insecticides, however many effective insecticides, such as parathion-ethyl, parathion-methyl and gamma-HCH, are no longer allowed in many areas of the world due to environmental, human safety, and regulatory issues. Additionally, other chemical insecticides have been shown not to be sufficiently efficacious against at least some species of flea beetle (Andersen et al., 2006, J Econ Entomol, 99(3): 803-810; Tansey et al., 2009, J Appl Entomol, 133: 201-209). Finally, flea beetle resistance to the remaining effective chemical insecticides is a significant concern. Therefore, novel compositions for control of flea beetles are urgently needed.

RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed.

RNAi has been found to be useful for insect control of certain insect pests. RNAi strategies typically employ a synthesized, non-naturally occurring “interfering RNA”, or “interfering RNA molecule” which typically comprises at least a RNA fragment against a target gene, a spacer sequence, and a second RNA fragment which is complementary to the first, so that a double-stranded RNA structure can be formed. This non-natural double-stranded RNA molecule takes advantage of the native RNAi pathways in the insect to trigger down-regulation of target genes that may lead to the cessation of feeding and/or growth and may result in the death of the insect pest.

Although it is known in the literature that RNAi strategies focused on target genes can lead to an insecticidal effect in Diabrotica species, it is also known that not every target sequence is successful, and that an insecticidal effect cannot be predicted. The overwhelming majority of sequences complementary to corn rootworm DNAs are not lethal in species of corn rootworm when used as dsRNA or siRNA. For example, Baum et al. ((2007) Nature Biotechnology 25:1322-1326), describe the effects of inhibiting several WCR gene targets by RNAi. The authors report that of 290 dsRNAs tested, only 125 showed significant larval mortality and/or stunting at the dsRNA concentration of 5.2 ng/cm². Additionally, the dosage or quantity of a given dsRNA molecule required to confer significant insecticidal activity needs to be considered for the dsRNA molecule to be of commercial value for crop protection.

There is an ongoing need for compositions containing insecticidal active ingredients, and for methods of using such compositions, for instance for use in crop protection or insect-mediated disease control. Novel compositions are required to overcome the problem of resistance to existing insecticides and/or to help mitigate the development of resistance to existing transgenic plant approaches. Ideally such compositions have a high toxicity and are effective when ingested orally by the target pest and have applicability for use against both the larval and adult stages of the pest insect. Thus any invention which provided compositions in which any of these properties was enhanced would represent a step forward in the art.

### SUMMARY

The needs outlined above are met by the invention which, in various embodiments, provides new methods of controlling economically important insect pests. The invention in part comprises a method of inhibiting expression of one or more target genes and proteins in Coleopteran insect pests. Specifically, the invention comprises methods of modulating expression of one or more target genes in a species of insect that causes cessation of feeding, growth, development and reproduction, and eventually results in the death of the insect, where the insect is a flea beetle species of the Alticini tribe. In some embodiments, the insect is a species of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In embodiments, the insect is a species selected from the group consisting of Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle), and related species. The method comprises introduction of an interfering RNA molecule comprising a double-stranded RNA (dsRNA) or its modified forms such as small interfering RNA (siRNA) sequences, into cells or into the extracellular environment, such as the midgut, within a pest insect body wherein the dsRNA or siRNA enters the cells and inhibits expression of at least one or more target genes and wherein inhibition of the one or more target genes exerts a deleterious effect upon the pest insect. The interfering RNA molecule is non-naturally occurring. It is specifically contemplated that the methods and compositions of the invention will be useful in limiting or eliminating pest insect infestation in or on any plant by providing one or more compositions comprising interfering RNA molecules comprising dsRNA or siRNA molecules in the diet of the pest. The invention also provides interfering RNA molecules that when delivered to an insect pest inhibits, through a toxic effect, the ability of the insect pest to survive, grow, feed and/or reproduce, or to limit pest related damage or loss to crop plants. Such delivery may be through production of the interfering RNA in a transgenic plant, for example canola, or by topically applying a composition comprising the interfering RNA to a plant or plant seed, such as a canola plant or canola seed. Delivery may further be through contacting the insect with the interfering RNA, such as when the insect feeds on plant material comprising the interfering RNA, either because the plant material is expressing the interfering RNA through a transgenic approach, or because the plant material is coated with a composition comprising the interfering RNA. The interfering RNA may also be provided in an artificial insect diet which the insect then contacts by feeding. The interfering RNA molecule comprises a nucleotide sequence that is complementary to a nucleotide sequence of a mRNA transcribable from a target gene or a portion of a nucleotide sequence of a mRNA transcribable from a target gene of the pest insect and therefore inhibits expression of the target gene, which causes cessation of feeding, growth, development, reproduction and eventually results in death of the pest insect. The invention is further drawn to nucleic acid constructs, nucleic acid molecules and recombinant vectors that comprise or encode at least a fragment of one strand of an interfering RNA molecule of the invention. The invention also provides chimeric nucleic acid molecules comprising an antisense strand of a dsRNA of the interfering RNA operably associated with a plant microRNA precursor molecule. The invention also provides artificial plant microRNA precursors comprising an antisense strand of a dsRNA of an interfering RNA of the invention.

The invention further provides an interfering ribonucleic acid (RNA) molecule wherein the RNA comprises at least one dsRNA wherein the dsRNA is a region of double-stranded RNA comprising annealed complementary strands, one strand of which comprises a sequence of at least 19 contiguous nucleotides which is at least partially complementary to a target nucleotide sequence within a flea beetle target gene, and (i) is at least 85% identical to at least a 19 contiguous nucleotide fragment of SEQ ID NO: 253-378 or SEQ ID NO: 616-701, or the complement thereof; or (ii) comprises at least a 19 contiguous nucleotide fragment of SEQ ID NO: 409-612, or the complement thereof; or (iii) comprises at least a 19 contiguous nucleotide fragment of a nucleotide sequence encoding an amino acid sequence encoded by SEQ ID NO: 409-612, or the complement thereof, wherein the interfering RNA molecule has insecticidal activity on a Coleopteran plant pest. In some embodiments, the interfering molecule may comprise at least two dsRNAs, wherein each dsRNA comprises a sequence of nucleotides which is at least partially complementary to a target nucleotide sequence within the target gene. In further embodiments, each of the dsRNAs may comprise a different sequence of nucleotides which is complementary to a different target nucleotide sequence within the target gene.

The invention further provides compositions comprising one or more interfering RNA molecules comprising two or more of dsRNA molecules, wherein the two or more RNA molecules each comprise a different antisense strand, or comprising two or more nucleic acid constructs or nucleic acid molecules or artificial plant microRNA precursors of the invention.

The invention further provides insecticidal compositions for inhibiting the expression of a Coleopteran insect gene that comprises a dsRNA of the invention and an agriculturally acceptable carrier. In one embodiment, inhibition of the expression of a flea beetle gene described here leads to cessation of feeding and growth and ultimately results in the death of the flea beetle.

The invention is further drawn to transgenic plants which produce one or more interfering RNA molecules of the invention that are self-protected from insect feeding damage and to methods of using the plants alone or in combination with other insect control strategies to confer maximal insect control capabilities. Plants and/or plant parts producing one or more interfering RNA molecules of the invention or treated with a composition comprising one or more interfering RNA molecules of the invention are highly resistant to insect pest infestation. For example, economically important Coleopteran pests can be controlled by a plant that produces an interfering RNA molecule of the invention or by a plant or plant seed that is treated with a composition comprising an interfering RNA molecule of the invention.

The invention also provides a method of controlling a Coleopteran insect plant pest comprising contacting the Coleopteran insect with a nucleic acid molecule that is or is capable of producing an interfering RNA of the invention for inhibiting expression of a gene in the Coleopteran insect thereby controlling the Coleopteran insect.

In other aspects, the invention provides a method of reducing a flea beetle population on a transgenic plant expressing a second insecticidal agent, for example an insecticidal protein, in addition to an interfering RNA of the invention capable of inhibiting expression of an target gene in a flea beetle, thereby reducing the flea beetle population. The second insecticidal agent may be an insecticidal protein derived from Bacillus thuringiensis. A B. thuringiensis insecticidal protein can be any of a number of insecticidal proteins including but not limited to a Cry1 protein, a Cry3 protein, a Cry7 protein, a Cry8 protein, a Cry11 protein, a Cry22 protein, a Cry 23 protein, a Cry 36 protein, a Cry37 protein, a Cry34 protein together with a Cry35 protein, a binary insecticidal protein CryET33 and CryET34, a binary insecticidal protein TIC100 and TIC101, a binary insecticidal protein PS149B1, a VIP, a TIC900 or related protein, a TIC901, TIC1201, TIC407, TIC417, a modified Cry3A protein, or hybrid proteins or chimeras made from any of the preceding insecticidal proteins. The insecticidal protein may be any other insecticidal protein derived from B. thuringiensis known in the art to be insecticidal (see for example, Palma et al., 2014, Toxins 6: 3296-3325, and references within; Berry and Crickmore, 2017, J of Invertebrate Pathology 142: 16-22, and reference within).

In other embodiments, the second insecticidal agent may be derived from sources other than B. thuringiensis. The second insecticidal agent can be an agent selected from the group comprising a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, a Clostridium spp. (such as C. bifermentans) insecticidal protein, and a lignin. In other embodiments, the second agent may be at least one insecticidal protein derived from an insecticidal toxin complex (Tc) from Photorhabdus, Xenorhabus, Serratia, or Yersinia. In other embodiments, the insecticidal protein may be an ADP-ribosyltransferase derived from an insecticidal bacteria, such as Photorhabdus spp. In other embodiments, the insecticidal protein may be a VIP protein, such as VIP1 or VIP2 from B. cereus. In still other embodiments, the insecticidal protein may be a binary toxin derived from an insecticidal bacteria, such as ISP1A and ISP2A from B. laterosporous or BinA and BinB from L. sphaericus. In still other embodiments, the insecticidal protein may be engineered or may be a hybrid or chimera of any of the preceding insecticidal proteins.

In other aspects, the invention provides a method of reducing resistance development in a flea beetle population to an interfering RNA of the invention, the method comprising expressing in a transgenic plant fed upon by the flea beetle population an interfering RNA of the invention that is capable of inhibiting expression of a target gene in a larval and adult flea beetle, thereby reducing resistance development in the flea beetle population compared to a flea beetle population exposed to an interfering RNA capable of inhibiting expression of a flea beetle gene described herein in only the larval stage or adult stage of a flea beetle.

In other aspects, the invention provides a method of reducing the level of a target RNA transcribable from a flea beetle gene described herein in a flea beetle comprising contacting the flea beetle with a composition comprising an interfering RNA molecule of the invention, wherein the interfering RNA molecule reduces the level of the target RNA in a cell of the flea beetle.

In still other aspects, the invention provides a method of conferring flea beetle tolerance or Coleopteran plant pest tolerance to a plant, or part thereof, comprising introducing into the plant, or part thereof, an interfering RNA molecule, a dsRNA molecule, a nucleic acid construct, a chimeric nucleic acid molecule, an artificial plant microRNA precursor molecule and/or a composition of the invention, thereby conferring to the plant or part thereof tolerance to the flea beetle or Coleopteran plant pest.

In further aspects, the invention provides a method of reducing damage to the plant fed upon by a flea beetle, comprising introducing into cells of the plant an interfering RNA molecule, a dsRNA, a nucleic acid molecule, a nucleic acid construct, a chimeric nucleic acid molecule, an artificial plant microRNA precursor molecule and/or a composition of the invention, thereby reducing damage to the plant fed upon by a flea beetle.

In other aspects, the invention provides a method of producing a transgenic plant cell having toxicity to a Coleopteran insect, comprising introducing into a plant cell an interfering RNA molecule, a dsRNA, a nucleic acid molecule, a nucleic acid construct, a chimeric nucleic acid molecule, an artificial plant microRNA precursor molecule and/or a composition of the invention, thereby producing the transgenic plant cell having toxicity to the Coleopteran insect compared to a control plant cell.

In further aspects, the invention provides a method of producing a transgenic plant having enhanced tolerance to Coleopteran insect feeding damage, comprising introducing into a plant an interfering RNA molecule, a dsRNA, a nucleic acid molecule, a nucleic acid construct, a chimeric nucleic acid molecule, an artificial plant microRNA precursor molecule and/or a composition of the invention, thereby producing a transgenic plant having enhanced tolerance to Coleopteran insect feeding damage compared to a control plant.

In other aspects, the invention provides a method of enhancing control of a Coleopteran insect population comprising providing a transgenic plant or transgenic seed of the invention and applying to the transgenic plant or the transgenic seed a chemical pesticide that is insecticidal to a Coleopteran insect, thereby enhancing control of the Coleopteran insect population.

In other aspects, the invention provides a method of providing a canola grower with a means of controlling a Coleopteran insect pest population below an economic threshold in a canola crop comprising (a) selling or providing to the grower transgenic canola seed comprising a dsRNA, a nucleic acid molecule, a nucleic acid construct, a chimeric nucleic acid molecule, an artificial plant microRNA precursor molecule and/or a composition of the invention; and (b) advertising to the grower that the transgenic canola seed produces transgenic canola plants capable of controlling a Coleopteran insect pest population.

In another aspect, the invention provides a method of identifying an orthologous target gene for using as a RNAi strategy for the control of a different Coleopteran plant pest, said method comprising the steps of: a) producing a primer pair that will amplify a target selected from the group comprising or consisting of SEQ ID NO: 1-102, or a complement thereof; b) amplifying an orthologous target gene from a nucleic acid sample of the plant pest using the primer pair of step a); c) identifying a sequence of an orthologous target gene; d) producing an interfering RNA molecule, wherein the RNA comprises at least one dsRNA, wherein the dsRNA is a region of double-stranded RNA comprising annealed complementary strands, one strand of which comprises a sequence of at least 19 contiguous nucleotides which is at least partially complementary to the orthologous target nucleotide sequence within the target gene; and e) determining if the interfering RNA molecule of step (d) has insecticidal activity on the plant pest. If the interfering RNA has insecticidal activity on the plant pest target gene, an orthologous target gene for using in the control of a plant pest has been identified.

These and other aspects of the invention are set forth in more detail in the description of the invention below.

### BRIEF DESCRIPTION OF THE SEQUENCES IN THE SEQUENCE LISTING

The nucleic acid sequences listed in the accompanying sequence listing are shown using standard letter abbreviations for nucleotide bases, as defined in 37 C.F.R. § 1.822. The nucleic acid and amino acid sequences listed define molecules (i.e., polynucleotides and polypeptides, respectively) having the nucleotide and amino acid monomers arranged in the manner described. The nucleic acid and amino acid sequences listed also each define a genus of polynucleotides or polypeptides that comprise the nucleotide and amino acid monomers arranged in the manner described. In view of the redundancy of the genetic code, it will be understood that a nucleotide sequence including a coding sequence also describes the genus of polynucleotides encoding the same polypeptide as a polynucleotide consisting of the reference sequence. It will further be understood that an amino acid sequence describes the genus of polynucleotide ORFs encoding that polypeptide.

Only one strand of each nucleic acid sequence is shown, but the complementary strand is understood as included by any reference to the displayed strand. As the complement and reverse complement of a primary nucleic acid sequence are necessarily disclosed by the primary sequence, the complementary sequence and reverse complementary sequence reference to the nucleic acid sequence, unless it is explicitly stated to be otherwise (or it is clear to be otherwise from the context in which the sequence appears). Furthermore, as it is understood in the art that the nucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NOs: 1-17 are DNA coding sequences of the 17 Phyllotreta armoraciae target genes identified for assaying in the RNAi-based screen for insecticidal activity.

SEQ ID NOs: 18-34 are DNA coding sequences of the 17 Psylliodes chrysocephala target genes identified for assaying in the RNAi-based screen for insecticidal activity.

SEQ ID NOs: 35-51 are DNA coding sequences of the 21 Phyllotreta nemorum target genes identified for assaying in the RNAi-based screen for insecticidal activity.

SEQ ID NOs: 52-68 are DNA coding sequences of the 21 Phyllotreta striolata target genes identified for assaying in the RNAi-based screen for insecticidal activity.

SEQ ID NOs: 69-85 are DNA coding sequences of the 21 Phyllotreta atra target genes identified for assaying in the RNAi-based screen for insecticidal activity.

SEQ ID NOs: 86-102 are DNA coding sequences of the 21 Phyllotreta cruciferae target genes identified for assaying in the RNAi-based screen for insecticidal activity.

SEQ ID NOs: 103-119 are fragments of DNA coding sequences of P. armoraciae used to synthesize interfering RNA molecules to test for insecticidal activity in the RNAi-based screen.

SEQ ID NOs: 120-136 are fragments of DNA coding sequences of P. chrysocephala used to synthesize interfering RNA molecules to test for insecticidal activity in the RNAi-based screen.

SEQ ID NOs: 137-153 are fragments of DNA coding sequences of P. nemorum used to synthesize interfering RNA molecules to test for insecticidal activity in the RNAi-based screen.

SEQ ID NOs: 154-170 are fragments of DNA coding sequences of P. striolata used to synthesize interfering RNA molecules to test for insecticidal activity in the RNAi-based screen.

SEQ ID NOs: 171-187 are fragments of DNA coding sequences of P. atra used to synthesize interfering RNA molecules to test for insecticidal activity in the RNAi-based screen.

SEQ ID NOs: 188-204 are fragments of DNA coding sequences of P. cruciferae used to synthesize interfering RNA molecules to test for insecticidal activity in the RNAi-based screen.

SEQ ID NOs: 205-221 are DNA sequences of forward primers and SEQ ID NOs: 307-323 are DNA sequences of the corresponding reverse primers for producing the fragments of SEQ ID NOs: 103-119.

SEQ ID NOs: 222-238 are DNA sequences of forward primers and SEQ ID NOs: 324-340 are DNA sequences of the corresponding reverse primers for producing the fragments of SEQ ID NOs: 120-136.

SEQ ID NOs: 239-255 are DNA sequences of forward primers and SEQ ID NOs: 341-357 are DNA sequences of the corresponding reverse primers for producing the fragments of SEQ ID NOs: 137-153.

SEQ ID NOs: 256-272 are DNA sequences of forward primers and SEQ ID NOs: 358-374 are DNA sequences of the corresponding reverse primers for producing the fragments of SEQ ID NOs: 154-170.

SEQ ID NOs: 273-289 are DNA sequences of forward primers and SEQ ID NOs: 375-391 are DNA sequences of the corresponding reverse primers for producing the fragments of SEQ ID NOs: 171-187.

SEQ ID NOs: 290-306 are DNA sequences of forward primers and SEQ ID NOs: 392-408 are DNA sequences of the corresponding reverse primers for producing the fragments of SEQ ID NOs: 188-204.

SEQ ID NOs: 409-425 are the sense RNA sequences of the P. armoraciae DNA coding sequences of SEQ ID NOs: 1-17.

SEQ ID NOs: 426-442 are the sense RNA sequences of the P. chrysocephala DNA coding sequences of SEQ ID NOs: 18-34.

SEQ ID NOs: 443-459 are the sense RNA sequences of the P. nemorum DNA coding sequences of SEQ ID NOs: 35-51.

SEQ ID NOs: 460-476 are the sense RNA sequences of the P. striolata DNA coding sequences of SEQ ID NOs: 52-68.

SEQ ID NOs: 477-493 are the sense RNA sequences of the P. atra DNA coding sequences of SEQ ID NOs: 69-85

SEQ ID NOs: 494-510 are the sense RNA sequences of the P. cruciferae DNA coding sequences of SEQ ID NOs: 86-102.

SEQ ID NOs: 511-527 are the sense RNA sequences of the P. armoraciae DNA coding sequence fragments of SEQ ID NOs: 103-119.

SEQ ID NOs: 528-544 are the sense RNA sequences of the P. chrysocephala DNA coding sequence fragments of SEQ ID NOs: 120-136.

SEQ ID NOs: 545-561 are the sense RNA sequences of the P. nemorum DNA coding sequence fragments of SEQ ID NOs: 137-153.

SEQ ID NOs: 562-578 are the sense RNA sequences of the P. striolata DNA coding sequence fragments of SEQ ID NOs: 154-170.

SEQ ID NOs: 579-595 are the sense RNA sequences of the P. atra DNA coding sequence fragments of SEQ ID NOs: 171-187.

SEQ ID NOs: 596-612 are the sense RNA sequences of the P. cruciferae DNA coding sequence fragments of SEQ ID NOs: 188-204.

SEQ ID NOs: 613-629 are amino acid sequences encoded by the P. armoraciae DNA coding sequences of SEQ ID Nos: 1-17.

SEQ ID NOs: 630-646 are amino acid sequences encoded by the P. chrysocephala DNA coding sequences of SEQ ID Nos: 18-34.

SEQ ID NOs: 647-663 are amino acid sequences encoded by the P. nemorum DNA coding sequences of SEQ ID Nos: 35-51.

SEQ ID NOs: 664-680 are amino acid sequences encoded by the P. striolata DNA coding sequence of SEQ ID NOs: 52-68.

SEQ ID NOs: 681-697 are amino acid sequences encoded by the P. atra DNA coding sequence of SEQ ID NOs: 69-85.

SEQ ID NOs: 698-714 are amino acid sequences encoded by the P. cruciferae DNA coding sequence of SEQ ID NOs: 86-102.

### DETAILED DESCRIPTION

The following is a detailed description of the invention provided to aid those skilled in the art in practicing the invention. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments of the invention will be apparent to those skilled in the art in light of the instant disclosure, which do not depart from the invention. Hence, the following descriptions are intended to illustrate some particular embodiments of the invention, and not to exhaustively specify all permutations, combinations and variations thereof. Those of ordinary skill in the art will recognize that modifications and variations in the embodiments described herein may be made without departing from the spirit or scope of the invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

For clarity, certain terms used in the specification are defined and presented as follows:

As used herein, “a,” “an” or “the” can mean one or more than one. For example, “a cell” can mean a single cell or a multiplicity of cells.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.” A “coding sequence” is a nucleic acid sequence that is transcribed into RNA such as mRNA, rRNA, tRNA, snRNA, sense RNA or antisense RNA. Preferably the RNA is then translated in an organism to produce a protein.

The terms “sequence similarity” or “sequence identity” of nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using one of the following sequence comparison algorithms or by visual inspection. In certain embodiments, substantially identical sequences have at least about 60%, or at least about 70%, or at least about 80%, or even at least about 90% or 95% nucleotide or amino acid residue identity. In certain embodiments, substantial identity exists over a region of the sequences that is at least about 50 residues in length, or over a region of at least about 100 residues, or the sequences are substantially identical over at least about 150 residues. In further embodiments, the sequences are substantially identical when they are identical over the entire length of the coding regions.

The term “homology” in the context of the invention refers to the level of similarity between nucleic acid or amino acid sequences in terms of nucleotide or amino acid identity or similarity, respectively, i.e., sequence similarity or identity. Homology, homologue, and homologous also refers to the concept of similar functional properties among different nucleic acids or proteins. Homologues include genes that are orthologous and paralogous. Homologues can be determined by using the coding sequence for a gene, disclosed herein or found in appropriate database (such as that at NCBI or others) in one or more of the following ways. For an amino acid sequence, the sequences should be compared using algorithms (for instance see section on “identity” and “substantial identity”). For nucleotide sequences the sequence of one DNA molecule can be compared to the sequence of a known or putative homologue in much the same way. Homologues are at least 20% identical, or at least 30% identical, or at least 40% identical, or at least 50% identical, or at least 60% identical, or at least 70% identical, or at least 80% identical, or at least 88% identical, or at least 90% identical, or at least 92% identical, or at least 95% identical, across any substantial region of the molecule (DNA, RNA, or protein molecule).

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another widely used and accepted computer program for performing sequence alignments is CLUSTALW v1.6 (Thompson, et al. Nuc. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between a 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a polynucleotide will hybridize to its target sequence to a detectably greater degree than other sequences (e.g., at least 2-fold over background). Stringent conditions are sequence-dependent and will be different in different circumstances. By controlling the stringency of the hybridization and/or washing conditions, target polynucleotides can be identified which are 100% complementary to the probe (homologous probing). Alternatively, stringency conditions can be adjusted to allow some mismatching in sequences so that lower degrees of similarity are detected (heterologous probing). Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2×SSC (20×SSC=3.0 M NaCl/0.3 M trisodium citrate) at 50 to 55° C. Moderate stringency conditions detect sequences that share at least 80% sequence identity. Exemplary moderate stringency conditions include hybridization in 40 to 45% formamide, 1 M NaCl, 1% SDS at 37° C., and a wash in 0.5× to 1×SSC at 55 to 60° C. High stringency conditions detect sequences that share at least 90% sequence identity. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tm can be approximated from the equation of Meinkoth and Wahl (Anal. Biochem., 138:267-284, 1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., eds., Greene Publishing and Wiley-Interscience, New York (1995). Methods of stringent hybridization are known in the art which conditions can be calculated by means known in the art. This is disclosed in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000. Methods of determining percent sequence identity are known in the art, an example of which is the GCG computer sequence analysis software (GCG, Inc, Madison Wis.).

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions.

A nucleic acid sequence is “isocoding with” a reference nucleic acid sequence when the nucleic acid sequence encodes a polypeptide having the same amino acid sequence as the polypeptide encoded by the reference nucleic acid sequence.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “dsRNA” or “RNAi” refers to a polyribonucleotide structure formed either by a single self-complementary RNA strand or at least by two complementary RNA strands. The degree of complementary, in other words the % identity, need not necessarily be 100%. Rather, it must be sufficient to allow the formation of a double-stranded structure under the conditions employed. As used herein, the term “fully complementary” means that all the bases of the nucleotide sequence of the dsRNA are complementary to or ‘match’ the bases of the target nucleotide sequence. The term “at least partially complementary” means that there is less than a 100% match between the bases of the dsRNA and the bases of the target nucleotide sequence. The skilled person will understand that the dsRNA need only be at least partially complementary to the target nucleotide sequence in order to mediate down-regulation of expression of the target gene. It is known in the art that RNA sequences with insertions, deletions and mismatches relative to the target sequence can still be effective at RNAi. According to the current invention, it is preferred that the dsRNA and the target nucleotide sequence of the target gene share at least 80% or 85% sequence identity, preferably at least 90% or 95% sequence identity, or more preferably at least 97% or 98% sequence identity and still more preferably at least 99% sequence identity. Alternatively, the dsRNA may comprise 1, 2 or 3 mismatches as compared with the target nucleotide sequence over every length of 24 partially complementary nucleotides. It will be appreciated by the person skilled in the art that the degree of complementarity shared between the dsRNA and the target nucleotide sequence may vary depending on the target gene to be down-regulated or depending on the insect pest species in which gene expression is to be controlled.

It will be appreciated that the dsRNA may comprise or consist of a region of double-stranded RNA comprising annealed complementary strands, one strand of which, the sense strand, comprises a sequence of nucleotides at least partially complementary to a target nucleotide sequence within a target gene.

The target nucleotide sequence may be selected from any suitable region or nucleotide sequence of the target gene or RNA transcript thereof. For example, the target nucleotide sequence may be located within the 5′UTR or 3′UTR of the target gene or RNA transcript or within exonic or intronic regions of the gene. The skilled person will be aware of methods of identifying the most suitable target nucleotide sequences within the context of the full-length target gene. For example, multiple dsRNAs targeting different regions of the target gene can be synthesised and tested. Alternatively, digestion of the RNA transcript with enzymes such as RNAse H can be used to determine sites on the RNA that are in a conformation susceptible to gene silencing. Target sites may also be identified using in silico approaches, for example, the use of computer algorithms designed to predict the efficacy of gene silencing based on targeting different sites within the full-length gene.

Preferably, the % identity of a polyribonucleotide is determined by GAP (Needleman and Wunsch, 1970) analysis (GCG program) using the default settings, wherein the query sequence is at least about 21 to about 23 nucleotides in length, and the GAP analysis aligns the two sequences over a region of at least about 21 nucleotides. In another embodiment, the query sequence is at least 150 nucleotides in length, and the GAP analysis aligns the two sequences over a region of at least 150 nucleotides. In a further embodiment, the query sequence is at least 300 nucleotides in length and the GAP analysis aligns the two sequences over a region of at least 300 nucleotides. In yet another embodiment, the query sequence corresponds to the full length of the target RNA, for example mRNA, and the GAP analysis aligns the two sequences over the full length of the target RNA.

Conveniently, the dsRNA can be produced from a single open reading frame in a recombinant host cell, wherein the sense and anti-sense sequences are flanked by an unrelated sequence which enables the sense and anti-sense sequences to hybridize to form the dsRNA molecule with the unrelated sequence forming a loop structure. In some embodiments, the sense strand and antisense strand can be made without an open reading frame to ensure that no protein will be made in the transgenic host cell. The two strands can also be expressed separately as two transcripts, one encoding the sense strand and one encoding the antisense strand.

RNA duplex formation can be initiated either inside or outside the cell. The dsRNA can be partially or fully double-stranded. The RNA can be enzymatically or chemically synthesized, either in vitro or in vivo.

The dsRNA need not be full length relative to either the primary transcription product or fully processed RNA. It is well-known in the art that small dsRNA of about 19-23 bp in length can be used to trigger gene silencing of a target gene. Generally, higher identity can be used to compensate for the use of a shorter sequence. Furthermore, the dsRNA can comprise single stranded regions as well, e.g., the dsRNA can be partially or fully double stranded. The double stranded region of the dsRNA can have a length of at least about 19 to about 23 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs, up to a molecule that is double stranded for its full length, corresponding in size to a full length target RNA molecule. Bolognesi et al (2012, PLOS One, 7(10): e47534) teach that dsRNAs greater than or equal to about 60 bp are required for biological activity in artificial diet bioassays with Southern Corn Rootworm (SCR; Diabrotica undecimpunctata howardii).

Mao et al (2007, Nature Biotechnology, 35(11): 1307-1313) teach a transgenic plant expressing a dsRNA construct against a target gene (CYP6AE14) of an insect pest (cotton bollworm, Helicoverpa armigera). Insects feeding on the transgenic plant have small RNAs of about 19-23 bp in size of the target gene in their midgut, with a corresponding reduction in CYP6AE14 transcripts and protein. This suggests that the small RNAs were efficacious in reducing expression of the target gene in the insect pest. Therefore, small RNAs of about 19 bp, about 20 bp, about 21 bp, about 22 bp, about 23 bp, about 24 bp, about 25 bp, about 26 bp, about 27 bp, about 28 bp, about 29 bp, or about 30 bp may be efficacious in reducing expression of the target gene in an insect pest.

Alternatively, the dsRNA may comprise a target dsRNA of at least 19 base pairs, and the target dsRNA may be within a dsRNA “carrier” or “filler” sequence. For example, Bolognesi et al (2012) show that a 240 bp dsRNA encompassing a target dsRNA, which comprised a 21 bp contiguous sequence with 100% identity to the target sequence, had biological activity in bioassays with Southern Corn Rootworm. The target dsRNA may have a length of at least 19 to about 25 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs. Combined with the carrier dsRNA sequence, the dsRNA of the target sequence and the carrier dsRNA may have a total length of at least about 50 to about 100 base pairs, optionally a sequence of about 100 to about 200 base pairs, optionally a sequence of about 200 to about 500, and optionally a sequence of about 500 to about 1000 or more base pairs.

The dsRNA can contain known nucleotide analogs or modified backbone residues or linkages, which are synthetic, naturally occurring, and non-naturally occurring. Examples of such analogs include, without limitation, phosphorothioates, phosphoramidates, methyl phosphonates, chiralmethyl phosphonates and 2-O-methyl ribonucleotides.

As used herein, the term “specifically reduce the level of a target RNA and/or the production of a target protein encoded by the RNA”, and variations thereof, refers to the sequence of a portion of one strand of the dsRNA being sufficiently identical to the target RNA such that the presence of the dsRNA in a cell reduces the steady state level and/or the production of said RNA. In many instances, the target RNA will be mRNA, and the presence of the dsRNA in a cell producing the mRNA will result in a reduction in the production of said protein. Preferably, this accumulation or production is reduced at least 10%, more preferably at least 50%, even more preferably at least 75%, yet even more preferably at least 95% and most preferably 100%, when compared to a wild-type cell.

The consequences of inhibition can be confirmed by examination of the outward properties of the cell or organism or by biochemical techniques such as, but not limited to, Northern hybridization, reverse transcription, gene expression monitoring with a microarray, antibody binding, enzyme linked immunosorbent assay (ELISA), Western blotting, radioimmunoassay (RIA), and other immunoassays.

The interfering RNAs of the current invention may comprise one dsRNA or multiple dsRNAs, wherein each dsRNA comprises or consists of a sequence of nucleotides which is at least partially complementary to a target nucleotide sequence within the target gene and that functions upon uptake by an insect pest species to down-regulate expression of said target gene. Concatemeric RNA constructs of this type are described in WO2006/046148. In the context of the present invention, the term ‘multiple’ means at least two, at least three, at least four, etc and up to at least 10, 15, 20 or at least 30. In one embodiment, the interfering RNA comprises multiple copies of a single dsRNA i.e. repeats of a dsRNA that binds to a particular target nucleotide sequence within a specific target gene. In another embodiment, the dsRNAs within the interfering RNA comprise or consist of different sequences of nucleotides complementary to different target nucleotide sequences. It should be clear that combinations of multiple copies of the same dsRNA combined with dsRNAs binding to different target nucleotide sequences are within the scope of the current invention.

The dsRNAs may be arranged as one contiguous region of the interfering RNA or may be separated by the presence of linker sequences. The linker sequence may comprise a short random nucleotide sequence that is not complementary to any target nucleotide sequences or target genes. In one embodiment, the linker is a conditionally self-cleaving RNA sequence, preferably a pH-sensitive linker or a hydrophobic-sensitive linker. In one embodiment, the linker comprises a sequence of nucleotides equivalent to an intronic sequence. Linker sequences of the current invention may range in length from about 1 base pair to about 10000 base pairs, provided that the linker does not impair the ability of the interfering RNA to down-regulate the expression of target gene(s).

In addition to the dsRNA(s) and any linker sequences, the interfering RNA of the invention may comprise at least one additional polynucleotide sequence. In different embodiments of the invention, the additional sequence is chosen from (i) a sequence capable of protecting the interfering RNA against RNA processing, (ii) a sequence affecting the stability of the interfering RNA, (iii) a sequence allowing protein binding, for example to facilitate uptake of the interfering RNA by cells of the insect pest species, (iv) a sequence facilitating large-scale production of the interfering RNA, (v) a sequence which is an aptamer that binds to a receptor or to a molecule on the surface of the insect pest cells to facilitate uptake, or (vi) a sequence that catalyses processing of the interfering RNA within the insect pest cells and thereby enhances the efficacy of the interfering RNA. Structures for enhancing the stability of RNA molecules are well known in the art and are described further in WO2006/046148.

The interfering RNA may contain DNA bases, non-natural bases or non-natural backbone linkages or modifications of the sugar-phosphate backbone, for example to enhance stability during storage or enhance resistance to degradation by nucleases. Furthermore, the interfering RNA may be produced chemically or enzymatically by one skilled in the art through manual or automated reactions. Alternatively, the interfering RNA may be transcribed from a polynucleotide encoding the same. Thus, provided herein is an isolated polynucleotide encoding any of the interfering RNAs of the current invention.

The term “plant microRNA precursor molecule” as used herein describes a small (^{˜}70-300 nt) non-coding RNA sequence that is processed by plant enzymes to yield a ^{˜}19-24 nucleotide product known as a mature microRNA sequence. The mature sequences have regulatory roles through complementarity to messenger RNA (mRNA). The term “artificial plant microRNA precursor molecule” describes the non-coding miRNA precursor sequence prior to processing that is employed as a backbone sequence for the delivery of a siRNA molecule via substitution of the endogenous native miRNA/miRNA* duplex of the miRNA precursor molecule with that of a non-native, heterologous miRNA (amiRNA/amiRNA*; e.g. siRNA/siRNA*) that is then processed into the mature miRNA sequence with the siRNA sequence.

In the context of the invention, the term “toxic” used to describe a dsRNA of the invention means that the dsRNA molecules of the invention and combinations of such dsRNA molecules function as orally active insect control agents that have a negative effect on an insect. When a composition of the invention is delivered to the insect, the result is typically death of the insect, or the insect does not feed upon the source that makes the composition available to the insect. Such a composition may be a transgenic plant expressing the dsRNA of the invention.

To “control” or “controlling” insects means to inhibit, through a toxic effect, the ability of one or more insect pests to survive, grow, feed, and/or reproduce, or to limit insect-related damage or loss in crop plants. To “control” insects may or may not mean killing the insects, although it preferably means killing the insects. A composition that controls a target insect has insecticidal activity against the target insect.

To “deliver” or “delivering” a composition or dsRNA means that the composition or dsRNA comes in contact with an insect, resulting in a toxic effect and control of the insect. The composition or dsRNA can be delivered in many recognized ways, e.g., orally by ingestion by the insect via transgenic plant expression, formulated composition(s), sprayable composition(s), a bait matrix, or any other art-recognized toxicant delivery system.

The term “insect” as used herein includes any organism now known or later identified that is classified in the animal kingdom, phylum Arthropoda, class Insecta, including but not limited to insects in the orders Coleoptera (beetles), Lepidoptera (moths, butterflies), Diptera (flies), Protura, Collembola (springtails), Diplura, Microcoryphia (jumping bristletails), Thysanura (bristletails, silverfish), Ephemeroptera (mayflies), Odonata (dragonflies, damselflies), Orthoptera (grasshoppers, crickets, katydids), Phasmatodea (walkingsticks), Grylloblattodea (rock crawlers), Mantophasmatodea, Dermaptera (earwigs), Plecoptera (stoneflies), Embioptera (web spinners), Zoraptera, Isoptera (termites), Mantodea (mantids), Blattodea (cockroaches), Hemiptera (true bugs, cicadas, leafhoppers, aphids, scales), Thysanoptera (thrips), Psocoptera (book and bark lice), Phthiraptera (lice; including but not limited to suborders Amblycera, Ischnocera and Anoplura), Neuroptera (lacewings, owlflies, mantispids, antlions), Hymenoptera (bees, ants, wasps), Trichoptera (caddisflies), Siphonaptera (fleas), Mecoptera (scorpion flies), Strepsiptera (twisted-winged parasites), and any combination thereof.

A “life stage of an Alticini insect” or “flea beetle life stage” means the egg, larval, pupal or adult developmental form of an insect of the Alticini tribe, namely a species of flea beetle.

“Effective insect-controlling amount” of “insecticidally effective amount” means that concentration of dsRNA that inhibits, through a toxic effect, the ability of insects to survive, grow, feed and/or reproduce, or to limit insect-related damage or loss in crop plants. “Insecticidally effective amount” may or may not mean a concentration that kills the insects, although it preferably means that it kills the insects. In some embodiments, application of an insecticidally effective amount of the polynucleotide, such as a dsRNA molecule, to a plant improves the plant's resistance to infestation by the insect. In some embodiments, application of an insecticidally effective amount of the polynucleotide, such as a dsRNA molecule, to a crop plant improves yield (e.g., increased biomass, increased seed or fruit production, or increased oil, starch, sugar, or protein content) of that crop plant, in comparison to a crop plant not treated with the polynucleotide. While there is no upper limit on the concentrations and dosages of a polynucleotide as described herein that can be useful in the methods and compositions provided herein, lower effective concentrations and dosages will generally be sought for efficiency and economy.

Non-limiting embodiments of effective amounts of a polynucleotide include a range from about 10 nano grams per milliliter to about 100 micrograms per milliliter of a polynucleotide in a liquid form sprayed on a plant, or from about 10 milligrams per acre to about 100 grams per acre of polynucleotide applied to a field of plants, or from about 0.001 to about 0.1 microgram per milliliter of polynucleotide in an artificial diet for feeding the insect. Where compositions as described herein are topically applied to a plant, the concentrations can be adjusted in consideration of the volume of spray or treatment applied to plant leaves or other plant part surfaces, such as flower petals, stems, tubers, fruit, anthers, pollen, leaves, roots, or seeds. In one embodiment, a useful treatment for herbaceous plants using 25-mer polynucleotides is about 1 nanomole (nmol) of polynucleotides per plant, for example, from about 0.05 to 1 nmol polynucleotides per plant. Other embodiments for herbaceous plants include useful ranges of about 0.05 to about 100 nmol, or about 0.1 to about 20 nmol, or about 1 nmol to about 10 nmol of polynucleotides per plant. In certain embodiments, about 40 to about 50 nmol of a single-stranded polynucleotide as described herein are applied. In certain embodiments, about 0.5 nmol to about 2 nmol of a dsRNA as described herein is applied. In certain embodiments, a composition containing about 0.5 to about 2.0 milligrams per milliliter, or about 0.14 milligrams per milliliter of a dsRNA (or a single-stranded 21-mer) as described herein is applied. In certain embodiments, a composition of about 0.5 to about 1.5 milligrams per milliliter of a dsRNA polynucleotide as described herein of about 50 to about 200 or more nucleotides is applied. In certain embodiments, about 1 nmol to about 5 nmol of a dsRNA as described herein is applied to a plant. In certain embodiments, the polynucleotide composition as topically applied to the plant contains at least one polynucleotide as described herein at a concentration of about 0.01 to about 10 milligrams per milliliter, or about 0.05 to about 2 milligrams per milliliter, or about 0.1 to about 2 milligrams per milliliter. Very large plants, trees, or vines can require correspondingly larger amounts of polynucleotides. When using long dsRNA molecules that can be processed into multiple oligonucleotides (e.g., multiple triggers encoded by a single recombinant DNA molecule as disclosed herein) lower concentrations can be used. Non-limiting examples of effective polynucleotide treatment regimes include a treatment of between about 0.1 to about 1 nmol of polynucleotide molecule per plant, or between about 1 nmol to about 10 nmol of polynucleotide molecule per plant, or between about 10 nmol to about 100 nmol of polynucleotide molecule per plant.

Crops of useful plants that may be protected according to this aspect of the invention include crop plants and/or ornamental plants. Crop plants include for example, members of the Solanaceae, Brassicacae, Labiatae and Fabaceae families, and flowering ornamental plants include in particular members of the Rubiaceae. In some embodiments, the crop a plant in the family Brassicaceae, including a Brassica species selected from the group consisting of B. napus, B. juncea, B. carinata, B. rapa, B. oleracea, B. rupestris, B. septiceps, B. nigra, B. narinosa, B. perviridus, B. tournefortii, and B. fructiculosas. In other embodiments, the plant is selected from the group consisting of Glycine max, Linum usitatissimum, Zea mays, Carthamus tinctorius, Helianthus annuus, Nicotiana tabacum, Arabidopsis thaliana, Betholettia excelsa, Ricinus communis, Cocus nucifera, Coriandrum sativum, Gossypium spp., Arachis hypogaea, Simmondsia chinensis, Solanum tuberosum, Elaeis guineensis, Olea europaea, Oryza sativa, Cucurbita maxim, Hordeum vulgare, and Triticum aestivum. In some embodiments the following ornamental plants may be protected against attack/infestation from flea beetles: Gardenia spp, Rothmannia spp, and ornamental Brassicacae.

Crops of useful plants are to be understood as including those which are/have been made tolerant to herbicides or classes of herbicide and/or insecticide or classes of insecticide, and/or which have acquired a so-called “output” trait (e.g. improved storage staibilty, higher nutritional value, improved yield etc.) by conventional plant-breeding or genetic engineering methods. Examples of useful plants that have been rendered tolerant to herbicides by genetic engineering methods include e.g. glyphosate- and glufosinate resistant varieties available under the trade names RoundupReady® and LibertyLink®, (e.g. RoundupReady® Canola and LibertyLink® Canola). An example of a crop that has been rendered tolerant to imidazolininone herbicides (e.g. imazamox) by conventional breeding methods includes Clearfield® summer rape (canola). Thus useful plants include those where the plants are transgenic, or where the plants have inherited a trait as a consequence of the introduction at least one transgene in their lineage.

As shown herein, the dsRNA molecules of the invention are surprisingly effective at controlling insects, namely flea beetles, in the Alticini tribe. The control of such insects is particularly important where it has been found that such insects exhibit resistance (or tolerance) to the insecticides that have hitherto been used for their control. Thus the methods of the invention not only have applicability against Alticini tribe members that are sensitive to insecticides other than the compositions of the invention, but also against Alticini tribe members that are resistant to insecticides, in particular Alticini tribe members resistant to pyrethroid and/or organophosphate pesticides.

In preferred embodiments of the aspects of the invention discussed herein, a composition of the invention is used to control insects of the tribe Alticini, commonly known as flea beetles.

Adult flea beetles damage plants by chewing numerous small holes in the leaves. Flea beetles can be the most detrimental to seedlings, however when the populations are high, flea beetles can defoliate and kill entire established plants. Flea beetles may also transmit viral and bacterial diseases which further affect the health and yield of the crop plants. Thus, in further aspects the invention provides methods of increasing the yield from crops of useful plants that are under attack by flea beetles from the tribe Alticini and/or maintaining yield or reducing yield loss from crops of useful plants that susceptible to attack by flea beetles of the tribe Alticini.

The methods of the present invention may be used to control all insects of the tribe Alticini. In some embodiments, the methods of the present invention may be used to control all insects of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In particular, methods of the invention may be used in the control of the following species: Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle). In further embodiments the methods of the invention will be used to control P. chrysocephala, P. nemorum, and P. striolata.

Methods include those developed for specific flea beetle pests for a given plant, e.g., wherein the plant is a potato plant and the insect is Epitrix cucumeris (potato flea beetle). In some embodiments, specific target genes are identified as targets for RNAi-mediated control in a given insect species. Various embodiments of the method include those wherein (a) the insect is Phyllotreta armoraciae and the target gene has a DNA sequence selected from the group consisting of SEQ ID NOs:1-17; (b) the insect is Psylliodes chrysocephala and the target gene has a DNA sequence selected from the group consisting of SEQ ID NOs: 18-34; (c) the insect is Phyllotreta nemorum and the target gene has a DNA sequence selected from the group consisting of SEQ ID NOs: 35-51; (d) the insect is Phyllotreta striolata and the target gene has a DNA sequence selected from the group consisting of SEQ ID NOs: 52-68; (e) the insect is Phyllotreta atra and the target gene has a DNA sequence selected from the group consisting of SEQ ID NOs: 69-85; or (f) the insect is Phyllotreta cruciferae and the target gene has a DNA sequence selected from the group consisting of SEQ ID NOs: 86-102.

The term “agrochemically active ingredient” refers to chemicals and/or biological compositions, such as those described herein, which are effective in killing, preventing, or controlling the growth of undesirable pests, such as, plants, insects, mice, microorganism, algae, fungi, bacteria, and the like (such as pesticidally active ingredients). An interfering RNA molecule of the invention is an agrochemically active ingredient.

An “agriculturally acceptable carrier” includes adjuvants, mixers, enhancers, etc. beneficial for application of an active ingredient, such as an interfering RNA molecule of the invention. Suitable carriers should not be phytotoxic to valuable crops, particularly at the concentrations employed in applying the compositions in the presence of crops, and should not react chemically with the compounds of the active ingredient herein, namely an interfering RNA of the invention, or other composition ingredients. Such mixtures can be designed for application directly to crops, or can be concentrates or formulations which are normally diluted with additional carriers and adjuvants before application. They may include inert or active components and can be solids, such as, for example, dusts, granules, water dispersible granules, or wettable powders, or liquids, such as, for example, emulsifiable concentrates, solutions, emulsions or suspensions. Suitable agricultural carriers may include liquid carriers, for example water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers may include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like.

It is recognized that the polynucleotides comprising sequences encoding the silencing element can be used to transform organisms to provide for host organism production of these components, and further used for subsequent application of the host organism to the environment of the target pest(s). In this manner, the combination of polynucleotides encoding the silencing element may be introduced via a suitable vector into a microbial host, and said host applied to the environment, or to plants or animals.

For the present invention, an agriculturally acceptable carrier may also include non-pathogenic, attenuated strains of microorganisms, which carry the insect control agent, namely an interfering RNA molecule of the invention. In this case, the microorganisms carrying the interfering RNA may also be referred to as insect control agents. The microorganisms may be engineered to express a nucleotide sequence of a target gene to produce interfering RNA molecules comprising RNA sequences homologous or complementary to RNA sequences typically found within the cells of an insect. Exposure of the insects to the microorganisms result in ingestion of the microorganisms and down-regulation of expression of target genes mediated directly or indirectly by the interfering RNA molecules or fragments or derivatives thereof.

Further, microbial hosts that are known to occupy the “phytosphere” (phylloplane, phyllosphere, rhizosphere, and/or rhizoplana) of one or more crops of interest may be selected. These microorganisms are selected so as to be capable of successfully competing in the particular environment with the wild-type microorganisms, provide for stable maintenance and expression of the sequences encoding the interfering RNA molecule of the invention, and desirably, provide for improved protection of the components from environmental degradation and inactivation.

Such microorganisms include bacteria, algae, and fungi. Of particular interest are microorganisms such as bacteria, e.g., Pseudomonas, Erwinia, Serratia, Klebsiella, Escherichia, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes; fungi, particularly yeast, e.g., Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacteria spp., Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans.

A number of ways are available for introducing the polynucleotide comprising the silencing element into the microbial host under conditions that allow for stable maintenance and expression of such nucleotide encoding sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

Transcriptional and translational regulatory signals include, but are not limited to, promoters, transcriptional initiation start sites, operators, activators, enhancers, other regulatory elements, ribosomal binding sites, an initiation codon, termination signals, and the like. Methods for the production of expression constructs comprising such regulatory signals are well known in the art; see for example Sambrook et al. (2000); Molecular Cloning: A Laboratory Manual (3rd ed.; Cold Spring Harbor Laboratory Press, Plainview, N.Y.); Davis et al. (1980) Advanced Bacterial Genetics (Cold Spring Harbor Laboratory, Cold Spring Harbor, N.Y.); and the references cited therein.

Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast such as Saccharomyces and Schizosaccharomyces; and Basidiomycetes yeast, such as Rhodotorula, Aureobasidium, Sporobolomyces, and the like.

Characteristics of particular interest in selecting a host cell for purposes of the invention include ease of introducing the coding sequence into the host, availability of expression systems, efficiency of expression, RNA stability in the host, and the presence of auxiliary genetic capabilities. Characteristics of interest for use as a pesticide microcapsule include protective qualities, such as thick cell walls, pigmentation, and intracellular packaging or formation of inclusion bodies; leaf affinity; lack of mammalian toxicity; attractiveness to pests for ingestion; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonas fluorescens, Saccharomyces cerevisiae, Bacillus thuringiensis, Escherichia coli, Bacillus subtilis, and the like.

The sequences encoding the interfering RNA molecules encompassed by the invention can be introduced into microorganisms that multiply on plants (epiphytes) to deliver these components to potential target pests. Epiphytes, for example, can be gram-positive or gram-negative bacteria.

An interfering RNA molecule of the invention can be fermented in a bacterial host and the resulting bacteria processed, and used as a microbial spray in the same manner that Bacillus thuringiensis strains have been used as insecticidal sprays. Any suitable microorganism can be used for this purpose. Pseudomonas spp. have been used to express Bacillus thuringiensis endotoxins as encapsulated proteins and the resulting cells processed and sprayed as an insecticide (Gaertner et al. 1993. Advanced Engineered Pesticides, ed. L. Kim (Marcel Decker, Inc.). E. coli is also well-known in the art for expressing molecules of interest as part during a fermentation process. In some embodiments, the resulting bacteria is processed by heat inactivation. In some embodiments, heat inactivation kills the bacteria but does not degrade the produced RNA molecules. The resulting compositions may then be formulated in accordance with conventional techniques for application to the environment hosting a target pest, e.g., soil, water, and foliage of plants.

Alternatively, the components of the invention are produced by introducing heterologous genes into a cellular host. Expression of the heterologous sequences results, directly or indirectly, in the intracellular production of the silencing element. These compositions may then be formulated in accordance with conventional techniques for application to the environment hosting a target pest, e.g., soil, water, and foliage of plants.

The transformed microorganisms carrying an interfering RNA molecule of the invention may also be referred to as insect control agents. The microorganisms may be engineered to express a nucleotide sequence of a target gene to produce interfering RNA molecules comprising RNA sequences homologous or complementary to RNA sequences typically found within the cells of an insect. Exposure of the insects to the microorganisms result in ingestion of the microorganisms and down-regulation of expression of target genes mediated directly or indirectly by the interfering RNA molecules or fragments or derivatives thereof.

In the present invention, a transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present invention (i.e., at least one interfering RNA molecule) are normally applied in the form of compositions and can be applied to the crop area, plant, or seed to be treated. For example, the compositions may be applied to grain in preparation for or during storage in a grain bin or silo, etc. The compositions may be applied simultaneously or in succession with other compounds. Methods of applying an active ingredient or a composition that contains at least one interfering RNA molecule include, but are not limited to, foliar application, seed coating, and soil application. The number of applications and the rate of application depend on the intensity of infestation by the corresponding pest.

The compositions comprising an interfering RNA molecule of the invention can be in a suitable form for direct application or as a concentrate of primary composition that requires dilution with a suitable quantity of water or other dilutant before application. The compositions (including the transformed microorganisms) can be applied to the environment of an insect pest such as a flea beetle, for example an insect species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, or Psylliodes punctulata (hop flea beetle), by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged.

Application is of the compounds of the invention is preferably to a crop of canola plants, the locus thereof or propagation material thereof. Preferably application is to a crop of canola plants or the locus thereof, more preferably to a crop of canola plants. Application may be before infestation or when the pest is present. Application of the compounds of the invention can be performed according to any of the usual modes of application, e.g. foliar, drench, soil, in furrow etc.

The compounds of the invention may be applied in combination with an attractant. An attractant is a chemical that causes the insect to migrate towards the location of application. Suitable attractants may include glucose, saccharose, salt, glutamate (e.g. Aji-no-Moto™), and citric acid (e.g. Orobor™).

An attractant may be premixed with the compound of the invention prior to application, e.g. as a ready-mix or tank-mix, or by simultaneous application or sequential application to the plant. Suitable rates of attractants are for example 0.02 kg/ha-3 kg/ha.

The compositions can conveniently contain another insecticide if this is thought necessary. In an embodiment of the invention, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, a herbicide, an insecticide, a fertilizer, in an inert carrier, and dead cells of a Bacillus strain or live or dead cells of transformed microorganisms of the invention.

In another embodiment, the interfering RNA molecules may be encapsulated in a synthetic matrix such as a polymer and applied to the surface of a host such as a plant. Ingestion of the host cells by an insect permits delivery of the insect control agents to the insect and results in down-regulation of a target gene in the host.

A composition of the invention, for example a composition comprising an interfering RNA molecule of the invention and an agriculturally acceptable carrier, may be used in conventional agricultural methods. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, plants, and the like.

A composition of the invention may be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating techniques and machines, such as fluidized bed techniques, the roller mill method, rotostatic seed treaters, and drum coaters.

In order to apply an active ingredient to insects of the Alticini tribe and/or crops of useful plants as required by the methods of the invention said active ingredient may be used in pure form or, more typically, formulated into a composition which includes, in addition to said active ingredient, a suitable inert diluent or carrier and optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). SFAs include non-ionic, cationic and/or anionic surfactants, as well as surfactant mixtures. Thus in further embodiments according to any aspect of the invention mentioned hereinbefore, the active ingredient will be in the form of a composition additionally comprising an agriculturally acceptable carrier or diluent.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing the active ingredient with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing the active ingredient with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of the active ingredient and one or more powdered solid diluents or carriers, or from pre-formed blank granules by absorbing the active ingredient (or a solution thereof, in a suitable agent) in a porous granular material (such as pumice, attapulgite clays, fuller's earth, kieselguhr, diatomaceous earths or ground corn cobs) or by adsorbing the active ingredient (or a solution thereof, in a suitable agent) on to a hardcore material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving the active ingredient in water or an organic solvent, such as a ketone, alcohol or glycol ether. These solutions may contain a surface active agent (for example to improve water dilution or prevent crystallisation in a spray tank). Emulsifiable concentrates (EC) or oil-in-water emulsions (EW) may be prepared by dissolving the active ingredient in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 15060 and SOLVESSO 200; SOLVESSO is a Registered TradeMark), ketones (such as cyclohexanone or methylcyclohexanone) and alcohols (such as benzyl alcohol, furfuryl alcohol or butanol), N-alkylpyrrolidones (such as N-methylpyrrolidoneor N-octylpyrrolidone), dimethyl amides of fatty acids (such as C8-C10 fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EW s include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SF As, to produce spontaneously a thermodynamically stable isotropic liquid formulation. The active ingredient is present initially in either the water or the solvent/SPA blend. Suitable solvents for use in MEs include those hereinbefore described for use in ECs or in EWs. A ME may be either an oil-in-water or a water-in-oil system (which system is present may be determined by conductivity measurements) and may be suitable for mixing water-soluble and oil-soluble pesticides in the same formulation. A ME is suitable for dilution into water, either remaining as a microemulsion or forming a conventional oil-in-water emulsion.

Suspension concentrates (SC) may comprise aqueous or non-aqueous suspensions of finely divided insoluble solid particles the active ingredient. SCs may be prepared by ball or bead milling the solid active ingredient in a suitable medium, optionally with one or more dispersing agents, to produce a fine particle suspension of the compound. One or more wetting agents may be included in the composition and a suspending agent may be included to reduce the rate at which the particles settle. Alternatively, the active ingredient may be dry milled and added to water, containing agents hereinbefore described, to produce the desired end product.

Aerosol formulations comprise the active ingredient and a suitable propellant (for example n-butane). Active ingredients may also be dissolved or dispersed in a suitable medium (for example water or a water miscible liquid, such as n-propanol) to provide compositions for use in non-pressurized, hand-actuated spray pumps. The active ingredient may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains the active ingredient and, optionally, a carrier or diluent therefor. The polymeric shell may be produced by either an interfacial polycondensation reaction or by a coacervation procedure. The compositions may provide for controlled release of the compound of the active ingredient. Active ingredients may also be formulated in a biodegradable polymeric matrix to provide a slow, controlled release of the compound. A composition may include one or more additives to improve the biological performance of the composition (for example by improving wetting, retention or distribution on surfaces; resistance to rain on treated surfaces; or uptake or mobility of the active ingredient. Such additives include surface active agents, spray additives based on oils, for example certain mineral oils, natural plant oils (such as soy bean and rape seed oil) and/or modified plant oils (e.g. esterified plant oils), and blends of these with other bio-enhancing adjuvants (ingredients which may aid or modify the action of the active ingredient. Where the active ingredient described herein is employed in methods of protecting crops of useful plants, methods of enhancing/maintaining yield and/or methods of increasing/maintaining pollination in crops of useful plants, it is preferred that said active ingredient (or compositions containing such active ingredient) is applied to the crop of useful plants at the flower-bud stage. In particular for crops of useful plants wherein said plants have yellow flowers, (e.g. oilseed rape, mustard etc.) it is preferred that the application occurs at the green to yellow bud stage.

Wetting agents, dispersing agents and emulsifying agents may be surface SFAs of the cationic, anionic, amphoteric or non-ionic type. Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of the invention may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of the invention may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of the invention (for example 0.0001 to 10%, by weight) depending upon the purpose for which they are to be used.

A compound of the invention may be used in mixtures with fertilizers (for example nitrogen-, potassium- or phosphorus-containing fertilizers). Suitable formulation types include granules of fertilizer. The mixtures preferably contain up to 25% by weight of the compound of the invention.

The invention therefore also provides a fertilizer composition comprising a fertilizer and a compound of the invention.

The compositions of this invention may contain other compounds having biological activity, for example micronutrients or compounds having fungicidal activity or which possess plant growth regulating, herbicidal, insecticidal, nematicidal or acaricidal activity.

The compound of the invention may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of the invention; or help to overcome or prevent the development of resistance to individual components.

Compositions of the invention include those prepared by premixing prior to application, e.g. as a readymix or tankmix, or by simultaneous application or sequential application to the plant.

In some embodiments, an acceptable agricultural carrier is a formulation useful for applying the composition comprising the interfering RNA molecule to a plant or seed. In some embodiments, the interfering RNA molecules are stabilized against degradation because of their double stranded nature and the introduction of Dnase/Rnase inhibitors. For example, dsRNA or siRNA can be stabilized by including thymidine or uridine nucleotide 3′ overhangs. The dsRNA or siRNA contained in the compositions of the invention can be chemically synthesized at industrial scale in large amounts. Methods available would be through chemical synthesis or through the use of a biological agent.

In other embodiments the formulation comprises a transfection promoting agent. In other embodiments, the transfection promoting agent is a lipid-containing compound. In further embodiments, the lipid-containing compound is selected from the group consisting of; Lipofectamine, Cellfectin, DMRIE-C, DOTAP and Lipofectin. In another embodiment, the lipid-containing compound is a Tris cationic lipid.

In some embodiments, the formulation further comprises a nucleic acid condensing agent. The nucleic acid condensing agent can be any such compound known in the art. Examples of nucleic acid condensing agents include, but are not limited to, spermidine (N-[3-aminopropyl]-1,4-butanediamine), protamine sulphate, poly-lysine as well as other positively charged peptides. In some embodiments, the nucleic acid condensing agent is spermidine or protamine sulfate.

In still further embodiments, the formulation further comprises buffered sucrose or phosphate buffered saline.

“Expression cassette” as used herein means a nucleic acid sequence capable of directing expression of a particular nucleic acid sequence in an appropriate host cell, comprising a promoter operably linked to the nucleic acid sequence of interest which is operably linked to termination signal sequences. It also typically comprises sequences required for proper translation of the nucleic acid sequence. The expression cassette comprising the nucleic acid sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleic acid sequence in the expression cassette may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

A “gene” is a defined region that is located within a genome and that, besides the aforementioned coding sequence, comprises other, primarily regulatory nucleic acid sequences responsible for the control of the expression, that is to say the transcription and translation, of the coding portion. A gene may also comprise other 5′ and 3′ untranslated sequences and termination sequences. Further elements that may be present are, for example, introns.

As used herein, the term “grower” means a person or entity that is engaged in agriculture, raising living organisms, such as crop plants, for example canola, for food, feed or raw materials.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

“Insecticidal” is defined as a toxic biological activity capable of controlling insects, preferably by killing them.

An “isolated” nucleic acid molecule or nucleotide sequence or nucleic acid construct or dsRNA molecule or protein of the invention is generally exists apart from its native environment and is therefore not a product of nature. An isolated nucleic acid molecule or nucleotide sequence or nucleic acid construct or dsRNA molecule or protein may exist in a purified form or may exist in a non-native environment such as, for example, a recombinant host or host cell such as a transgenic plant or transgenic plant cell.

In the context of the invention, a number in front of the suffix “mer” indicates a specified number of subunits. When applied to RNA or DNA, this specifies the number of bases in the molecule. For example, a 19 nucleotide subsequence of an mRNA having the sequence AGUCGUGUUGGUCCUCGGG is a “19-mer” of SEQ ID NO: 506.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A flea beetle “transcriptome” is a collection of all or nearly all the ribonucleic acid (RNA) transcripts in a flea beetle cell.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The nomenclature used herein for DNA or RNA bases and amino acids is as set forth in 37 C.F.R. § 1.822.

The invention is based on the unexpected discovery that double stranded RNA (dsRNA) or small interfering RNAs (siRNA) designed to target a mRNA transcribable from the flea beetle genes described herein are toxic to the flea beetle pest and can be used to control flea beetle or Coleopteran infestation of a plant and impart to a transgenic plant tolerance to a flea beetle or Coleopteran infestation. Thus, in one embodiment, the invention provides a double stranded RNA (dsRNA) molecule comprising a sense strand and an antisense strand, wherein a nucleotide sequence of the antisense strand is complementary to a portion of a mRNA polynucleotide transcribable from a flea beetle gene described in the present disclosure, wherein the dsRNA molecule is toxic to a flea beetle or Coleopteran plant pest.

It is known in the art that dsRNA molecules that are not perfectly complementary to a target sequence (for example, having only 95% identity to the target gene) are effective to control Coleopteran pests (see, for example, Narva et al., U.S. Pat. No. 9,012,722). The invention provides an interfering RNA molecule comprising at least one dsRNA, where the dsRNA is a region of double-stranded RNA comprising annealed at least partially complementary strands. One strand of the dsRNA comprises a sequence of at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides which is at least partially complementary to a target nucleotide sequence within a flea beetle target gene. The interfering RNA molecule (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 409-612, or the complement thereof, or (iv) can hybridize under stringent conditions to a polynucleotide selected from the group consisting of SEQ ID NO: 409-612, and the complements thereof, wherein the interfering RNA molecule has insecticidal activity on a Coleopteran plant pest, for example a flea beetle insect.

In some embodiments, the interfering RNA molecule comprises at least two dsRNAs, wherein each dsRNA comprises a sequence of nucleotides which is at least partially complementary to a target nucleotide sequence within the target gene. In some embodiments, each of the dsRNAs comprise a different sequence of nucleotides which is complementary to a different target nucleotide sequence within the target gene. In other embodiments, each of the dsRNAs comprise a different sequence of nucleotides which is complementary to a target nucleotide sequence within two different target genes.

In some embodiments, the interfering RNA molecule comprises a dsRNA that can comprise, consist essentially of or consist of from at least 18 to about 25 consecutive nucleotides (e.g. 18, 19, 20, 21, 22, 23, 24, 25, 26, 27, 28, or 29) to at least about 300 consecutive nucleotides. Additional nucleotides can be added at the 3′ end, the 5′ end or both the 3′ and 5′ ends to facilitate manipulation of the dsRNA molecule but that do not materially affect the basic characteristics or function of the dsRNA molecule in RNA interference (RNAi).

In some embodiments, the interfering RNA molecule comprises a dsRNA which comprises an antisense strand that is complementary to at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 consecutive nucleotides of SEQ ID NO: 409-612, or the complement thereof. In other embodiments, the portion of dsRNA comprises, consists essentially of or consists of at least from 19, 20 or 21 consecutive nucleotides to at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 consecutive nucleotides of SEQ ID NO: 409-612, or the complement thereof.

In other embodiments, an interfering RNA molecule of the invention comprises a dsRNA which comprises, consists essentially of or consists of any 21-mer subsequence of SEQ ID NO: 409-510 consisting of N to N+20 nucleotides, or any complement thereof. For example, an interfering RNA molecule of the invention comprises a dsRNA which comprises, consist essentially of or consists of any 21-mer subsequence of SEQ ID NO: 409, wherein N is nucleotide 1 to nucleotide 784 of SEQ ID NO: 409, or any complement thereof. In other words, the portion of the mRNA that is targeted comprises any of the 784 21 consecutive nucleotide subsequences i.e. 21-mers) of SEQ ID NO: 409, or any of their complementing sequences. It will be recognized that these 784 21 consecutive nucleotide subsequences include all possible 21 consecutive nucleotide subsequences from SEQ ID NO: 409 and from SEQ ID NO: 511, and their complements, as SEQ ID NO's 409 and 511 are all to the same target, namely Pa1, which also may be referred to as a P. armoraciae ortholog of Rpn12. It will similarly be recognized that all 21-mer subsequences of SEQ ID NO: 409-510, and all complement subsequences thereof, include all possible 21 consecutive nucleotide subsequences of SEQ ID NO: 511-612, and the complement subsequences thereof.

Similarly, an interfering RNA molecule of the invention comprises a dsRNA which comprises, consist essentially of or consists of any 21-mer subsequence of SEQ ID NO: 410, wherein N is nucleotide 1 to nucleotide 2701 of SEQ ID NO: 2410, or any complement thereof. Another interfering RNA molecule of the invention comprises a dsRNA which comprises, consist essentially of or consists of any 21-mer subsequence of SEQ ID NO: 411, wherein N is nucleotide 1 to nucleotide 752 of SEQ ID NO: 411, or any complement thereof. Another interfering RNA molecule of the invention comprises a dsRNA which comprises, consist essentially of or consists of any 21-mer subsequence of SEQ ID NO: 412, wherein N is nucleotide 1 to nucleotide 811 of SEQ ID NO: 412, or any complement thereof. Another interfering RNA molecule of the invention comprises a dsRNA which comprises, consist essentially of or consists of any 21-mer subsequence of SEQ ID NO: 413, wherein N is nucleotide 1 to nucleotide 556 of SEQ ID NO: 413, or any complement thereof. Another interfering RNA molecule of the invention comprises a dsRNA which comprises, consist essentially of or consists of any 21-mer subsequence of SEQ ID NO: 414, wherein N is nucleotide 1 to nucleotide 442 of SEQ ID NO: 414, or any complement thereof. Another interfering RNA molecule of the invention comprises a dsRNA which comprises, consist essentially of or consists of any 21-mer subsequence of SEQ ID NO: 415, wherein N is nucleotide 1 to nucleotide 850 of SEQ ID NO: 415, or any complement thereof. Another interfering RNA molecule of the invention comprises a dsRNA which comprises, consist essentially of or consists of any 21-mer subsequence of SEQ ID NO: 416, wherein N is nucleotide 1 to nucleotide 352 of SEQ ID NO: 416, or any complement thereof. Another interfering RNA molecule of the invention comprises a dsRNA which comprises, consist essentially of or consists of any 21-mer subsequence of SEQ ID NO: 417, wherein N is nucleotide 1 to nucleotide 334 of SEQ ID NO: 417, or any complement thereof. Another interfering RNA molecule of the invention comprises a dsRNA which comprises, consist essentially of or consists of any 21-mer subsequence of SEQ ID NO: 418, wherein N is nucleotide 1 to nucleotide 355 of SEQ ID NO: 418, or any complement thereof. Another interfering RNA molecule of the invention comprises a dsRNA which comprises, consist essentially of or consists of any 21-mer subsequence of SEQ ID NO: 419, wherein N is nucleotide 1 to nucleotide 5674 of SEQ ID NO: 419, or any complement thereof. Another interfering RNA molecule of the invention comprises a dsRNA which comprises, consist essentially of or consists of any 21-mer subsequence of SEQ ID NO: 420, wherein N is nucleotide 1 to nucleotide 511 of SEQ ID NO: 420, or any complement thereof. Another interfering RNA molecule of the invention comprises a dsRNA which comprises, consist essentially of or consists of any 21-mer subsequence of SEQ ID NO: 421, wherein N is nucleotide 1 to nucleotide 388 of SEQ ID NO: 421, or any complement thereof. Another interfering RNA molecule of the invention comprises a dsRNA which comprises, consist essentially of or consists of any 21-mer subsequence of SEQ ID NO: 422, wherein N is nucleotide 1 to nucleotide 385 of SEQ ID NO: 422, or any complement thereof. Another interfering RNA molecule of the invention comprises a dsRNA which comprises, consist essentially of or consists of any 21-mer subsequence of SEQ ID NO: 423, wherein N is nucleotide 1 to nucleotide 400 of SEQ ID NO: 423, or any complement thereof. Another interfering RNA molecule of the invention comprises a dsRNA which comprises, consist essentially of or consists of any 21-mer subsequence of SEQ ID NO: 424, wherein N is nucleotide 1 to nucleotide 1702 of SEQ ID NO: 424, or any complement thereof. Another interfering RNA molecule of the invention comprises a dsRNA which comprises, consist essentially of or consists of any 21-mer subsequence of SEQ ID NO: 425, wherein N is nucleotide 1 to nucleotide 3973 of SEQ ID NO: 425, or any complement thereof. Another interfering RNA molecule of the invention comprises a dsRNA which comprises, consist essentially of or consists of any 21-mer subsequence of SEQ ID NO: 426, wherein N is nucleotide 1 to nucleotide 784 of SEQ ID NO: 426, or any complement thereof. Another interfering RNA molecule of the invention comprises a dsRNA which comprises, consist essentially of or consists of any 21-mer subsequence of SEQ ID NO: 427, wherein N is nucleotide 1 to nucleotide 436 of SEQ ID NO: 427, or any complement thereof. Another interfering RNA molecule of the invention comprises a dsRNA which comprises, consist essentially of or consists of any 21-mer subsequence of SEQ ID NO: 428, wherein N is nucleotide 1 to nucleotide 787 of SEQ ID NO: 428, or any complement thereof. Another interfering RNA molecule of the invention comprises a dsRNA which comprises, consist essentially of or consists of any 21-mer subsequence of SEQ ID NO: 429, wherein N is nucleotide 1 to nucleotide 805 of SEQ ID NO: 429, or any complement thereof. Another interfering RNA molecule of the invention comprises a dsRNA which comprises, consist essentially of or consists of any 21-mer subsequence of SEQ ID NO: 430, wherein N is nucleotide 1 to nucleotide 562 of SEQ ID NO: 430, or any complement thereof. Another interfering RNA molecule of the invention comprises a dsRNA which comprises, consist essentially of or consists of any 21-mer subsequence of SEQ ID NO: 431, wherein N is nucleotide 1 to nucleotide 439 of SEQ ID NO: 431, or any complement thereof. Another interfering RNA molecule of the invention comprises a dsRNA which comprises, consist essentially of or consists of any 21-mer subsequence of SEQ ID NO: 432, wherein N is nucleotide 1 to nucleotide 856 of SEQ ID NO: 432, or any complement thereof. Another interfering RNA molecule of the invention comprises a dsRNA which comprises, consist essentially of or consists of any 21-mer subsequence of SEQ ID NO: 433, wherein N is nucleotide 1 to nucleotide 352 of SEQ ID NO: 433, or any complement thereof. Another interfering RNA molecule of the invention comprises a dsRNA which comprises, consist essentially of or consists of any 21-mer subsequence of SEQ ID NO: 434, wherein N is nucleotide 1 to nucleotide 334 of SEQ ID NO: 434, or any complement thereof. Another interfering RNA molecule of the invention comprises a dsRNA which comprises, consist essentially of or consists of any 21-mer subsequence of SEQ ID NO: 435, wherein N is nucleotide 1 to nucleotide 355 of SEQ ID NO: 435, or any complement thereof. Another interfering RNA molecule of the invention comprises a dsRNA which comprises, consist essentially of or consists of any 21-mer subsequence of SEQ ID NO: 436, wherein N is nucleotide 1 to nucleotide 5674 of SEQ ID NO: 436, or any complement thereof. Another interfering RNA molecule of the invention comprises a dsRNA which comprises, consist essentially of or consists of any 21-mer subsequence of SEQ ID NO: 437, wherein N is nucleotide 1 to nucleotide 511 of SEQ ID NO: 437, or any complement thereof. Another interfering RNA molecule of the invention comprises a dsRNA which comprises, consist essentially of or consists of any 21-mer subsequence of SEQ ID NO: 438, wherein N is nucleotide 1 to nucleotide 388 of SEQ ID NO: 438, or any complement thereof. Another interfering RNA molecule of the invention comprises a dsRNA which comprises, consist essentially of or consists of any 21-mer subsequence of SEQ ID NO: 439, wherein N is nucleotide 1 to nucleotide 385 of SEQ ID NO: 439, or any complement thereof. Another interfering RNA molecule of the invention comprises a dsRNA which comprises, consist essentially of or consists of any 21-mer subsequence of SEQ ID NO: 440, wherein N is nucleotide 1 to nucleotide 400 of SEQ ID NO: 440, or any complement thereof. Another interfering RNA molecule of the invention comprises a dsRNA which comprises, consist essentially of or consists of any 21-mer subsequence of SEQ ID NO: 441, wherein N is nucleotide 1 to nucleotide 2047 of SEQ ID NO: 441, or any complement thereof. Another interfering RNA molecule of the invention comprises a dsRNA which comprises, consist essentially of or consists of any 21-mer subsequence of SEQ ID NO: 442, wherein N is nucleotide 1 to nucleotide 3964 of SEQ ID NO: 442, or any complement thereof. Another interfering RNA molecule of the invention comprises a dsRNA which comprises, consist essentially of or consists of any 21-mer subsequence of SEQ ID NO: 443, wherein N is nucleotide 1 to nucleotide 784 of SEQ ID NO: 443, or any complement thereof. Another interfering RNA molecule of the invention comprises a dsRNA which comprises, consist essentially of or consists of any 21-mer subsequence of SEQ ID NO: 444, wherein N is nucleotide 1 to nucleotide 436 of SEQ ID NO: 444, or any complement thereof. Another interfering RNA molecule of the invention comprises a dsRNA which comprises, consist essentially of or consists of any 21-mer subsequence of SEQ ID NO: 445, wherein N is nucleotide 1 to nucleotide 787 of SEQ ID NO: 445, or any complement thereof. Another interfering RNA molecule of the invention comprises a dsRNA which comprises, consist essentially of or consists of any 21-mer subsequence of SEQ ID NO: 446, wherein N is nucleotide 1 to nucleotide 811 of SEQ ID NO: 446, or any complement thereof. Another interfering RNA molecule of the invention comprises a dsRNA which comprises, consist essentially of or consists of any 21-mer subsequence of SEQ ID NO: 447, wherein N is nucleotide 1 to nucleotide 487 of SEQ ID NO: 447, or any complement thereof. Another interfering RNA molecule of the invention comprises a dsRNA which comprises, consist essentially of or consists of any 21-mer subsequence of SEQ ID NO: 448, wherein N is nucleotide 1 to nucleotide 406 of SEQ ID NO: 448, or any complement thereof. Another interfering RNA molecule of the invention comprises a dsRNA which comprises, consist essentially of or consists of any 21-mer subsequence of SEQ ID NO: 449, wherein N is nucleotide 1 to nucleotide 856 of SEQ ID NO: 449, or any complement thereof. Another interfering RNA molecule of the invention comprises a dsRNA which comprises, consist essentially of or consists of any 21-mer subsequence of SEQ ID NO: 450, wherein N is nucleotide 1 to nucleotide 251 of SEQ ID NO: 450, or any complement thereof. Another interfering RNA molecule of the invention comprises a dsRNA which comprises, consist essentially of or consists of any 21-mer subsequence of SEQ ID NO: 451, wherein N is nucleotide 1 to nucleotide 334 of SEQ ID NO: 451, or any complement thereof. Another interfering RNA molecule of the invention comprises a dsRNA which comprises, consist essentially of or consists of any 21-mer subsequence of SEQ ID NO: 452, wherein N is nucleotide 1 to nucleotide 355 of SEQ ID NO: 452, or any complement thereof. Another interfering RNA molecule of the invention comprises a dsRNA which comprises, consist essentially of or consists of any 21-mer subsequence of SEQ ID NO: 453, wherein N is nucleotide 1 to nucleotide 5674 of SEQ ID NO: 453, or any complement thereof. Another interfering RNA molecule of the invention comprises a dsRNA which comprises, consist essentially of or consists of any 21-mer subsequence of SEQ ID NO: 454, wherein N is nucleotide 1 to nucleotide 511 of SEQ ID NO: 454, or any complement thereof. Another interfering RNA molecule of the invention comprises a dsRNA which comprises, consist essentially of or consists of any 21-mer subsequence of SEQ ID NO: 455, wherein N is nucleotide 1 to nucleotide 388 of SEQ ID NO: 455, or any complement thereof. Another interfering RNA molecule of the invention comprises a dsRNA which comprises, consist essentially of or consists of any 21-mer subsequence of SEQ ID NO: 456, wherein N is nucleotide 1 to nucleotide 385 of SEQ ID NO: 456, or any complement thereof. Another interfering RNA molecule of the invention comprises a dsRNA which comprises, consist essentially of or consists of any 21-mer subsequence of SEQ ID NO: 457, wherein N is nucleotide 1 to nucleotide 400 of SEQ ID NO: 457, or any complement thereof. Another interfering RNA molecule of the invention comprises a dsRNA which comprises, consist essentially of or consists of any 21-mer subsequence of SEQ ID NO: 458, wherein N is nucleotide 1 to nucleotide 1189 of SEQ ID NO: 458, or any complement thereof. Another interfering RNA molecule of the invention comprises a dsRNA which comprises, consist essentially of or consists of any 21-mer subsequence of SEQ ID NO: 459, wherein N is nucleotide 1 to nucleotide 3514 of SEQ ID NO: 459, or any complement thereof. Another interfering RNA molecule of the invention comprises a dsRNA which comprises, consist essentially of or consists of any 21-mer subsequence of SEQ ID NO: 460, wherein N is nucleotide 1 to nucleotide 784 of SEQ ID NO: 460, or any complement thereof. Another interfering RNA molecule of the invention comprises a dsRNA which comprises, consist essentially of or consists of any 21-mer subsequence of SEQ ID NO: 461, wherein N is nucleotide 1 to nucleotide 436 of SEQ ID NO: 461, or any complement thereof. Another interfering RNA molecule of the invention comprises a dsRNA which comprises, consist essentially of or consists of any 21-mer subsequence of SEQ ID NO: 462, wherein N is nucleotide 1 to nucleotide 787 of SEQ ID NO: 462, or any complement thereof. Another interfering RNA molecule of the invention comprises a dsRNA which comprises, consist essentially of or consists of any 21-mer subsequence of SEQ ID NO: 463, wherein N is nucleotide 1 to nucleotide 811 of SEQ ID NO: 463, or any complement thereof. Another interfering RNA molecule of the invention comprises a dsRNA which comprises, consist essentially of or consists of any 21-mer subsequence of SEQ ID NO: 464, wherein N is nucleotide 1 to nucleotide 556 of SEQ ID NO: 464, or any complement thereof. Another interfering RNA molecule of the invention comprises a dsRNA which comprises, consist essentially of or consists of any 21-mer subsequence of SEQ ID NO: 465, wherein N is nucleotide 1 to nucleotide 439 of SEQ ID NO: 465, or any complement thereof. Another interfering RNA molecule of the invention comprises a dsRNA which comprises, consist essentially of or consists of any 21-mer subsequence of SEQ ID NO: 466, wherein N is nucleotide 1 to nucleotide 856 of SEQ ID NO: 466, or any complement thereof. Another interfering RNA molecule of the invention comprises a dsRNA which comprises, consist essentially of or consists of any 21-mer subsequence of SEQ ID NO: 467, wherein N is nucleotide 1 to nucleotide 352 of SEQ ID NO: 467, or any complement thereof. Another interfering RNA molecule of the invention comprises a dsRNA which comprises, consist essentially of or consists of any 21-mer subsequence of SEQ ID NO: 468, wherein N is nucleotide 1 to nucleotide 334 of SEQ ID NO: 468, or any complement thereof. Another interfering RNA molecule of the invention comprises a dsRNA which comprises, consist essentially of or consists of any 21-mer subsequence of SEQ ID NO: 469, wherein N is nucleotide 1 to nucleotide 355 of SEQ ID NO: 469, or any complement thereof. Another interfering RNA molecule of the invention comprises a dsRNA which comprises, consist essentially of or consists of any 21-mer subsequence of SEQ ID NO: 470, wherein N is nucleotide 1 to nucleotide 5260 of SEQ ID NO: 470, or any complement thereof. Another interfering RNA molecule of the invention comprises a dsRNA which comprises, consist essentially of or consists of any 21-mer subsequence of SEQ ID NO: 471, wherein N is nucleotide 1 to nucleotide 511 of SEQ ID NO: 471, or any complement thereof. Another interfering RNA molecule of the invention comprises a dsRNA which comprises, consist essentially of or consists of any 21-mer subsequence of SEQ ID NO: 472, wherein N is nucleotide 1 to nucleotide 388 of SEQ ID NO: 472, or any complement thereof. Another interfering RNA molecule of the invention comprises a dsRNA which comprises, consist essentially of or consists of any 21-mer subsequence of SEQ ID NO: 473, wherein N is nucleotide 1 to nucleotide 385 of SEQ ID NO: 473, or any complement thereof. Another interfering RNA molecule of the invention comprises a dsRNA which comprises, consist essentially of or consists of any 21-mer subsequence of SEQ ID NO: 474, wherein N is nucleotide 1 to nucleotide 400 of SEQ ID NO: 474, or any complement thereof. Another interfering RNA molecule of the invention comprises a dsRNA which comprises, consist essentially of or consists of any 21-mer subsequence of SEQ ID NO: 475, wherein N is nucleotide 1 to nucleotide 2044 of SEQ ID NO: 475, or any complement thereof. Another interfering RNA molecule of the invention comprises a dsRNA which comprises, consist essentially of or consists of any 21-mer subsequence of SEQ ID NO: 476, wherein N is nucleotide 1 to nucleotide 3511 of SEQ ID NO: 476, or any complement thereof. Another interfering RNA molecule of the invention comprises a dsRNA which comprises, consist essentially of or consists of any 21-mer subsequence of SEQ ID NO: 477, wherein N is nucleotide 1 to nucleotide 784 of SEQ ID NO: 477, or any complement thereof. Another interfering RNA molecule of the invention comprises a dsRNA which comprises, consist essentially of or consists of any 21-mer subsequence of SEQ ID NO: 478, wherein N is nucleotide 1 to nucleotide 412 of SEQ ID NO: 478, or any complement thereof. Another interfering RNA molecule of the invention comprises a dsRNA which comprises, consist essentially of or consists of any 21-mer subsequence of SEQ ID NO: 479, wherein N is nucleotide 1 to nucleotide 676 of SEQ ID NO: 479, or any complement thereof. Another interfering RNA molecule of the invention comprises a dsRNA which comprises, consist essentially of or consists of any 21-mer subsequence of SEQ ID NO: 480, wherein N is nucleotide 1 to nucleotide 811 of SEQ ID NO: 480, or any complement thereof. Another interfering RNA molecule of the invention comprises a dsRNA which comprises, consist essentially of or consists of any 21-mer subsequence of SEQ ID NO: 481, wherein N is nucleotide 1 to nucleotide 556 of SEQ ID NO: 481, or any complement thereof. Another interfering RNA molecule of the invention comprises a dsRNA which comprises, consist essentially of or consists of any 21-mer subsequence of SEQ ID NO: 482, wherein N is nucleotide 1 to nucleotide 439 of SEQ ID NO: 482, or any complement thereof. Another interfering RNA molecule of the invention comprises a dsRNA which comprises, consist essentially of or consists of any 21-mer subsequence of SEQ ID NO: 483, wherein N is nucleotide 1 to nucleotide 856 of SEQ ID NO: 483, or any complement thereof. Another interfering RNA molecule of the invention comprises a dsRNA which comprises, consist essentially of or consists of any 21-mer subsequence of SEQ ID NO: 484, wherein N is nucleotide 1 to nucleotide 352 of SEQ ID NO: 484, or any complement thereof. Another interfering RNA molecule of the invention comprises a dsRNA which comprises, consist essentially of or consists of any 21-mer subsequence of SEQ ID NO: 485, wherein N is nucleotide 1 to nucleotide 334 of SEQ ID NO: 485, or any complement thereof. Another interfering RNA molecule of the invention comprises a dsRNA which comprises, consist essentially of or consists of any 21-mer subsequence of SEQ ID NO: 486, wherein N is nucleotide 1 to nucleotide 361 of SEQ ID NO: 486, or any complement thereof. Another interfering RNA molecule of the invention comprises a dsRNA which comprises, consist essentially of or consists of any 21-mer subsequence of SEQ ID NO: 487, wherein N is nucleotide 1 to nucleotide 5668 of SEQ ID NO: 487, or any complement thereof. Another interfering RNA molecule of the invention comprises a dsRNA which comprises, consist essentially of or consists of any 21-mer subsequence of SEQ ID NO: 488, wherein N is nucleotide 1 to nucleotide 457 of SEQ ID NO: 488, or any complement thereof. Another interfering RNA molecule of the invention comprises a dsRNA which comprises, consist essentially of or consists of any 21-mer subsequence of SEQ ID NO: 489, wherein N is nucleotide 1 to nucleotide 388 of SEQ ID NO: 489, or any complement thereof. Another interfering RNA molecule of the invention comprises a dsRNA which comprises, consist essentially of or consists of any 21-mer subsequence of SEQ ID NO: 490, wherein N is nucleotide 1 to nucleotide 385 of SEQ ID NO: 490, or any complement thereof. Another interfering RNA molecule of the invention comprises a dsRNA which comprises, consist essentially of or consists of any 21-mer subsequence of SEQ ID NO: 491, wherein N is nucleotide 1 to nucleotide 400 of SEQ ID NO: 491, or any complement thereof. Another interfering RNA molecule of the invention comprises a dsRNA which comprises, consist essentially of or consists of any 21-mer subsequence of SEQ ID NO: 492, wherein N is nucleotide 1 to nucleotide 2044 of SEQ ID NO: 492, or any complement thereof. Another interfering RNA molecule of the invention comprises a dsRNA which comprises, consist essentially of or consists of any 21-mer subsequence of SEQ ID NO: 493, wherein N is nucleotide 1 to nucleotide 3970 of SEQ ID NO: 493, or any complement thereof. Another interfering RNA molecule of the invention comprises a dsRNA which comprises, consist essentially of or consists of any 21-mer subsequence of SEQ ID NO: 494, wherein N is nucleotide 1 to nucleotide 784 of SEQ ID NO: 494, or any complement thereof. Another interfering RNA molecule of the invention comprises a dsRNA which comprises, consist essentially of or consists of any 21-mer subsequence of SEQ ID NO: 495, wherein N is nucleotide 1 to nucleotide 436 of SEQ ID NO: 495, or any complement thereof. Another interfering RNA molecule of the invention comprises a dsRNA which comprises, consist essentially of or consists of any 21-mer subsequence of SEQ ID NO: 496, wherein N is nucleotide 1 to nucleotide 787 of SEQ ID NO: 496, or any complement thereof. Another interfering RNA molecule of the invention comprises a dsRNA which comprises, consist essentially of or consists of any 21-mer subsequence of SEQ ID NO: 497, wherein N is nucleotide 1 to nucleotide 811 of SEQ ID NO: 497, or any complement thereof. Another interfering RNA molecule of the invention comprises a dsRNA which comprises, consist essentially of or consists of any 21-mer subsequence of SEQ ID NO: 498, wherein N is nucleotide 1 to nucleotide 487 of SEQ ID NO: 498, or any complement thereof. Another interfering RNA molecule of the invention comprises a dsRNA which comprises, consist essentially of or consists of any 21-mer subsequence of SEQ ID NO: 499, wherein N is nucleotide 1 to nucleotide 439 of SEQ ID NO: 499, or any complement thereof. Another interfering RNA molecule of the invention comprises a dsRNA which comprises, consist essentially of or consists of any 21-mer subsequence of SEQ ID NO: 500, wherein N is nucleotide 1 to nucleotide 856 of SEQ ID NO: 500, or any complement thereof. Another interfering RNA molecule of the invention comprises a dsRNA which comprises, consist essentially of or consists of any 21-mer subsequence of SEQ ID NO: 501, wherein N is nucleotide 1 to nucleotide 352 of SEQ ID NO: 501, or any complement thereof. Another interfering RNA molecule of the invention comprises a dsRNA which comprises, consist essentially of or consists of any 21-mer subsequence of SEQ ID NO: 502, wherein N is nucleotide 1 to nucleotide 334 of SEQ ID NO: 502, or any complement thereof. Another interfering RNA molecule of the invention comprises a dsRNA which comprises, consist essentially of or consists of any 21-mer subsequence of SEQ ID NO: 503, wherein N is nucleotide 1 to nucleotide 355 of SEQ ID NO: 503, or any complement thereof. Another interfering RNA molecule of the invention comprises a dsRNA which comprises, consist essentially of or consists of any 21-mer subsequence of SEQ ID NO: 504, wherein N is nucleotide 1 to nucleotide 4528 of SEQ ID NO: 504, or any complement thereof. Another interfering RNA molecule of the invention comprises a dsRNA which comprises, consist essentially of or consists of any 21-mer subsequence of SEQ ID NO: 505, wherein N is nucleotide 1 to nucleotide 511 of SEQ ID NO: 505, or any complement thereof. Another interfering RNA molecule of the invention comprises a dsRNA which comprises, consist essentially of or consists of any 21-mer subsequence of SEQ ID NO: 506, wherein N is nucleotide 1 to nucleotide 388 of SEQ ID NO: 506, or any complement thereof. Another interfering RNA molecule of the invention comprises a dsRNA which comprises, consist essentially of or consists of any 21-mer subsequence of SEQ ID NO: 507, wherein N is nucleotide 1 to nucleotide 385 of SEQ ID NO: 507, or any complement thereof. Another interfering RNA molecule of the invention comprises a dsRNA which comprises, consist essentially of or consists of any 21-mer subsequence of SEQ ID NO: 508, wherein N is nucleotide 1 to nucleotide 400 of SEQ ID NO: 508, or any complement thereof. Another interfering RNA molecule of the invention comprises a dsRNA which comprises, consist essentially of or consists of any 21-mer subsequence of SEQ ID NO: 509, wherein N is nucleotide 1 to nucleotide 1939 of SEQ ID NO: 509, or any complement thereof. Another interfering RNA molecule of the invention comprises a dsRNA which comprises, consist essentially of or consists of any 21-mer subsequence of SEQ ID NO: 510, wherein N is nucleotide 1 to nucleotide 3970 of SEQ ID NO: 510, or any complement thereof.

In still other embodiments, the interfering RNA molecule of the invention comprises a dsRNA which comprises, consists essentially of or consists of SEQ ID NO: 409-612, or the complement thereof.

In other embodiments of the interfering RNA molecule of the invention, the nucleotide sequence of the antisense strand of a dsRNA of the invention comprises, consists essentially of or consists of the complementary RNA sequence of SEQ ID NO: 409-612. The nucleotide sequence of the antisense strand of a dsRNA of the invention can have one nucleotide at either the 3′ or 5′ end deleted or can have up to six nucleotides added at the 3′ end, the 5′ end or both, in any combination to achieve an antisense strand consisting essentially of any 19-mer, any 20-mer, or any 21-mer nucleotide sequence of SEQ ID NO: 409-612, as it would be understood that the deletion of the one nucleotide or the addition of up to the six nucleotides do not materially affect the basic characteristics or function of the double stranded RNA molecule of the invention. Such additional nucleotides can be nucleotides that extend the complementarity of the antisense strand along the target sequence and/or such nucleotides can be nucleotides that facilitate manipulation of the RNA molecule or a nucleic acid molecule encoding the RNA molecule, as would be known to one of ordinary skill in the art. For example, a TT overhang at the 3′ end may be present, which is used to stabilize the siRNA duplex and does not affect the specificity of the siRNA.

In some embodiments of this invention, the antisense strand of the double stranded RNA of the interfering RNA molecule can be fully complementary to the target RNA polynucleotide or the antisense strand can be substantially complementary or partially complementary to the target RNA polynucleotide. The dsRNA of the interfering RNA molecule may comprise a dsRNA which is a region of double-stranded RNA comprising substantially complementary annealed strands, or which is a region of double-stranded RNA comprising fully complementary annealed strands. By substantially or partially complementary is meant that the antisense strand and the target RNA polynucleotide can be mismatched at about 1, 2, 3, 4, 5, 6, 7, 8, 9, or 10 nucleotide pairings. Such mismatches can be introduced into the antisense strand sequence, e.g., near the 3′ end, to enhance processing of the double stranded RNA molecule by Dicer, to duplicate a pattern of mismatches in a siRNA molecule inserted into a chimeric nucleic acid molecule or artificial microRNA precursor molecule of this invention, and the like, as would be known to one of skill in the art. Such modification will weaken the base pairing at one end of the duplex and generate strand asymmetry, therefore enhancing the chance of the antisense strand, instead of the sense strand, being processed and silencing the intended gene (Geng and Ding “Double-mismatched siRNAs enhance selective gene silencing of a mutant ALS-causing Allele1” Acta Pharmacol. Sin. 29:211-216 (2008); Schwarz et al. “Asymmetry in the assembly of the RNAi enzyme complex” Cell 115:199-208 (2003)).

In some embodiments of this invention, the interfering RNA comprises a dsRNA which comprises a short hairpin RNA (shRNA) molecule. Expression of shRNA in cells is typically accomplished by delivery of plasmids or recombinant vectors, for example in transgenic plants such as transgenic canola.

The invention encompasses a nucleic acid construct comprising an interfering RNA of the invention. The invention further encompasses a nucleic acid molecule encoding at least one interfering molecule of the invention. The invention further encompasses a nucleic acid construct comprising at least one interfering molecule of the invention or comprising a nucleic acid molecule encoding the at least one interfering molecule of the invention. The invention further encompasses a nucleic acid construct wherein the nucleic acid construct is an expression vector. The invention further encompasses a recombinant vector comprising a regulatory sequence operably linked to a nucleotide sequence that encodes an interfering RNA molecule of the invention. A regulatory sequence may refer to a promoter, enhancer, transcription factor binding site, insulator, silencer, or any other DNA element involved in the expression of a gene.

The invention further encompasses chimeric nucleic acid molecules comprising an interfering RNA molecule with an antisense strand of a dsRNA operably linked with a plant microRNA precursor molecule. In some embodiments, the chimeric nucleic acid molecule comprises an antisense strand having the nucleotide sequence of any of the 21-mer subsequences complementary to SEQ ID NOs: 409-612, or any complement thereof, operably linked with a plant microRNA precursor molecule. In some embodiments, the plant microRNA precursor molecule is a canola microRNA precursor.

The use of artificial plant microRNAs to deliver a nucleotide sequence of interest (e.g an artificial miRNA; siRNA/siRNA*) into a plant is known in the art (see, e.g., Schwab et al. 2006. The Plant Cell 18:1121-1133 and Examples section herein). In the invention, the artificial microRNAs are chimeric or hybrid molecules, having a plant microRNA precursor backbone and an insect siRNA sequence inserted therein. As would be understood by one of ordinary skill in the art, it is typically desirable to maintain mismatches that normally occur in the plant microRNA precursor sequence in any nucleotide sequence that is substituted into the plant microRNA precursor backbone. In still other embodiments, the artificial plant microRNA precursor comprises portions of a canola microRNA precursor molecule. Any canola microRNA (miRNA) precursor is suitable for the compositions and methods of the invention. Non-limiting examples include miR156, miR159, miR166, miR167, miR168, miR169, miR171, miR172, miR319, miR390, miR393, miR394, miR395, miR396, miR397, miR398, miR399, miR408, miR482, miR528, miR529, miR827, miR838, miR1432, as well as any other plant miRNA precursors now known or later identified.

In some embodiments, the invention encompasses interfering RNA molecules, nucleic acid constructs, nucleic acid molecules or recombinant vectors comprising at least one strand of a dsRNA of an interfering RNA molecule of the invention, or comprising a chimeric nucleic acid molecule of the invention, or comprising an artificial plant microRNA of the invention. In some embodiments the nucleic acid construct comprises a nucleic acid molecule of the invention. In other embodiments, the nucleic acid construct is a recombinant expression vector.

In some embodiments, the interfering RNA molecules of the invention have insecticidal activity on an insect, namely a flea beetle, from the tribe Alticini. In some embodiments, the interfering RNA molecules of the present invention have activity on an insect which is species of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In embodiments, the insect is a species selected from the group consisting of Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle). In some embodiments, the coding sequence of the target gene comprises a sequence selected from the group comprising SEQ ID NO: 1-102.

In some embodiments, the invention encompasses a composition comprising one or more or two or more of the interfering RNA molecules of the invention. In some embodiments, the interfering RNA molecules are present on the same nucleic acid construct, on different nucleic acid constructs, or any combination thereof. For example, one interfering RNA molecule of the invention may be present on a nucleic acid construct, and a second interfering RNA molecule of the invention may be present on the same nucleic acid construct or on a separate, second nucleic acid construct. The second interfering RNA molecule of the invention may be to the same target gene or to a different target gene.

In some embodiments, the invention encompasses a composition comprising an interfering RNA molecule which comprises at least one dsRNA wherein the dsRNA is a region of double-stranded RNA comprising annealed complementary strands. One strand of the dsRNA comprises a sequence of at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides which is at least partially complementary to a target nucleotide sequence within a flea beetle target gene. The interfering RNA molecule (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 409-612, or the complement thereof, or (iv) can hybridize under stringent conditions to a polynucleotide selected from the group consisting of SEQ ID NO: 409-612, and the complements thereof.

In some embodiments, the invention encompasses compositions comprising an interfering RNA molecule comprising two or more dsRNAs, wherein the two or more dsRNAs each comprise a different antisense strand. In some embodiments the invention encompasses compositions comprising at least two more interfering RNA molecules, wherein the two or more interfering RNA molecules each comprise a dsRNA comprising a different antisense strand. The two or more interfering RNAs may be present on the same nucleic acid construct, on different nucleic acid constructs or any combination thereof. In other embodiments, the composition comprises a RNA molecule comprising an antisense strand consisting essentially of a nucleotide sequence comprising at least a 19 contiguous nucleotide fragment complementary to at least a 19 contiguous nucleotide fragment comprising the RNA sequence of SEQ ID NO: 409-612, and in some embodiments may further comprise an RNA molecule comprising an antisense strand consisting essentially of a second nucleotide sequence comprising at least a 19 contiguous nucleotide fragment complementary to at least a 19 contiguous nucleotide fragment of SEQ ID NO: 409-612; and in some embodiments may further comprise an RNA molecule comprising an antisense strand consisting essentially of a third nucleotide sequence comprising at least a 19 contiguous nucleotide fragment complementary to at least a 19 contiguous nucleotide fragment of SEQ ID NO: 409-612, and in some embodiments may further comprise an RNA molecule comprising an antisense strand consisting essentially of a fourth nucleotide sequence comprising at least a 19 contiguous nucleotide fragment complementary to at least a 19 contiguous nucleotide fragment of SEQ ID NO: 409-612, and in some embodiments may further comprise an RNA molecule comprising an antisense strand consisting essentially of a fifth nucleotide sequence comprising at least a 19 contiguous nucleotide fragment complementary to at least a 19 contiguous nucleotide fragment of SEQ ID NO: 409-612, and in some embodiments may further comprise an RNA molecule comprising an antisense strand consisting essentially of a sixth nucleotide sequence comprising at least a 19 contiguous nucleotide fragment complementary to at least a 19 contiguous nucleotide fragment of SEQ ID NO: 409-612, and in some embodiments may further comprise an RNA molecule comprising an antisense strand consisting essentially of a seventh nucleotide sequence comprising at least a 19 contiguous nucleotide fragment complementary to at least a 19 contiguous nucleotide fragment of SEQ ID NO: 409-612. In other embodiments, the composition may comprise two or more of the nucleic acid molecules, wherein the two or more nucleic acid molecules each encode a different interfering RNA molecule. In other embodiments, the composition may comprise two or more of the nucleic acid constructs, wherein the two or more nucleic acid constructs each comprise a nucleic acid molecule encoding a different interfering RNA.

In other embodiments, the composition comprises two or more nucleic acid constructs, two or more nucleic acid molecules, two or more chimeric nucleic acid molecules, two or more artificial plant microRNA precursors of the invention, wherein the two or more nucleic acid constructs, two or more nucleic acid molecules, two or more chimeric nucleic acid molecules, or two or more artificial plant microRNA precursors, each comprise a different antisense strand.

In some embodiments, the invention encompasses an insecticidal composition for inhibiting the expression of a flea beetle gene described herein, comprising an interfering RNA of the invention and an agriculturally acceptable carrier. In some embodiments, the acceptable agricultural carrier is a transgenic organism expressing an interfering RNA of the invention. In some embodiments the transgenic organism may be a transgenic plant expressing the interfering RNA of the invention that when fed upon by a target Coleopteran plant pest causes the target Coleopteran plant pest to stop feeding, growing or reproducing or causing death of the target Coleopteran plant pest. In other embodiments, the transgenic plant is a transgenic canola plant and the target pest is a flea beetle insect pest. In still other embodiments, the flea beetle insect pest is from the tribe Alticini. In other embodiments, the flea beetle is a species of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In embodiments, the insect is a species selected from the group consisting of Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).

In other embodiments, the transgenic organism is selected from, but not limited to, the group consisting of: yeast, fungi, algae, bacteria, virus or an arthropod expressing the interfering RNA molecule of the invention. In some embodiments, the transgenic organism is a virus, for example an insect baculovirus that expresses an interfering RNA molecule of the invention upon infection of an insect host. Such a baculovirus is likely more virulent against the target insect than the wildtype untransformed baculovirus. In other embodiments the transgenic organism is a transgenic bacterium that is applied to an environment where a target pest occurs or is known to have occurred. In some embodiments, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive interfering RNA molecule for the same purpose.

In some embodiments, an acceptable agricultural carrier is a formulation useful for applying the composition comprising the interfering RNA molecule to a plant or seed. In some embodiments, the interfering RNA molecules are stabilized against degradation because of their double stranded nature and the introduction of Dnase/Rnase inhibitors. For example, dsRNA or siRNA can be stabilized by including thymidine or uridine nucleotide 3′ overhangs. The dsRNA or siRNA contained in the compositions of the invention can be chemically synthesized at industrial scale in large amounts. Methods available would be through chemical synthesis or through the use of a biological agent.

In some embodiments, the invention encompasses transgenic plants, or parts thereof, comprising an interfering RNA molecule, a nucleic acid construct, a chimeric nucleic acid molecule, a artificial plant microRNA precursor molecule and/or a composition of the invention, wherein the transgenic plant has enhanced resistance to a Coleopteran insect or flea beetle as compared to a control plant. In other embodiments, the transgenic plant, or part thereof, is a transgenic canola plant, or part thereof. The invention further encompasses transgenic seed of the transgenic plants of the invention, wherein the transgenic seed comprises an interfering RNA molecule, a nucleic acid construct, a chimeric nucleic acid molecule, an artificial plant microRNA precursor molecule and/or a composition of the invention. In some embodiments the transgenic seed is a transgenic canola seed.

Transgenic plants expressing an interfering RNA of the invention are tolerant or resistant to attack by target insect pests. When the insect starts feeding on such a transgenic plant, it also ingests the expressed dsRNA or siRNA. This may deter the insect from further biting into the plant tissue or may even harm or kill the insect. A nucleic acid sequence encoding a dsRNA or siRNA of the invention is inserted into an expression cassette, which is then preferably stably integrated in the genome of the plant. The nucleic acid sequences of the expression cassette introduced into the genome of the plant are heterologous to the plant and non-naturally occurring. Plants transformed in accordance with the present invention may be monocots or dicots and include, but are not limited to, corn, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed (also referred to as canola), clover, tobacco, carrot, cotton, alfalfa, rice, potato, eggplant, cucumber, Arabidopsis, and woody plants such as coniferous and deciduous trees. In further embodiments, the transgenic plant is a transgenic canola plant.

Expression of the interfering RNA molecule in transgenic plants is driven by regulatory sequences comprising promoters that function in plants. The choice of promoter will vary depending on the temporal and spatial requirements for expression, and also depending on the insect target species. Thus, expression of the interfering RNAs of this invention in leaves, in stems, in inflorescences (e.g. anther, filament, pollen, style, petal, sepal, pedicel, stamen, etc.), in roots, and/or seedlings is contemplated. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the dsRNA or siRNA in the desired cell.

Promoters useful with the invention include, but are not limited to, those that drive expression of a nucleotide sequence constitutively, those that drive expression when induced, and those that drive expression in a tissue- or developmentally-specific manner. These various types of promoters are known in the art.

In some embodiments, tissue-specific/tissue-preferred promoters can be used. Tissue-specific or tissue-preferred expression patterns include, but are not limited to, green tissue specific or preferred, root specific or preferred, stem specific or preferred, and flower specific or preferred. In addition, promoters functional in plastids can be used. In some embodiments of the invention, inducible promoters can be used. In further aspects, the nucleotide sequences of the invention can be operably associated with a promoter that is wound inducible or inducible by pest or pathogen infection (e.g., a insect or nematode plant pest)

In some embodiments of the present invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

In some embodiments, a recombinant nucleic acid molecule of the invention can be an “expression cassette.” As used herein, “expression cassette” means a recombinant nucleic acid molecule comprising a nucleotide sequence of interest (e.g., the nucleotide sequences of the invention), wherein the nucleotide sequence is operably associated with at least a control sequence (e.g., a promoter). Thus, some embodiments of the invention provide expression cassettes designed to express nucleotides sequences encoding the dsRNAs or siRNAs of the invention. In this manner, for example, one or more plant promoters operably associated with one or more nucleotide sequences of the invention are provided in expression cassettes for expression in a canola plant, plant part and/or plant cell.

An expression cassette comprising a nucleotide sequence of interest may be chimeric, meaning that at least one of its components is heterologous with respect to at least one of its other components. An expression cassette may also be one that comprises a native promoter driving its native gene, however it has been obtained in a recombinant form useful for heterologous expression. Such usage of an expression cassette makes it so it is not naturally occurring in the cell into which it has been introduced.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

An expression cassette of the invention also can include polynucleotides that encode other desired traits. Such desired traits can be other polynucleotides which confer insect resistance, or which confer nematode resistance, or other agriculturally desirable traits. Such polynucleotides can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a single transgene can comprise multiple expression cassettes, such that multiple expression cassettes are introduced into the genome of a transformed cell at a single genomic location. Alternatively, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

Thus, an expression cassette can include a coding sequence for one or more polypeptides for agronomic traits that primarily are of benefit to a seed company, grower or grain processor. A polypeptide of interest can be any polypeptide encoded by a polynucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construct of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors of the invention may also comprise other selectable marker genes, for example, phosphomannose isomerase (pmi), which provides for positive selection of the transgenic plants as disclosed in U.S. Pat. Nos. 5,767,378 and 5,994,629, or phosphinotricin acetyltransferase (pat), which provides tolerance to the herbicide phosphinotricin (glufosinate). The choice of selectable marker is not, however, critical to the invention.

In other embodiments, a nucleic acid sequence of the invention is directly transformed into the plastid genome. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Nati. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransf erase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleic acid sequence of the present invention is inserted into a plastid-targeting vector and transformed into the plastid genome of a desired plant host. Plants homoplastic for plastid genomes containing a nucleic acid sequence of the present invention are obtained, and are preferentially capable of high expression of the nucleic acid sequence.

The compositions of the invention can also be combined with other biological control agents to enhance control of a Coleopteran insect or a flea beetle populations. Thus, the invention provides a method of enhancing control of a Coleopteran insect population or a flea beetle population by providing a transgenic plant that produces an interfering RNA of the invention and further comprises a polynucleotide that encodes a second insecticidal agent. The second insecticidal agent may be an insecticidal protein derived from Bacillus thuringiensis. A B. thuringiensis insecticidal protein can be any of a number of insecticidal proteins including but not limited to a Cry1 protein, a Cry3 protein, a Cry7 protein, a Cry8 protein, a Cry11 protein, a Cry22 protein, a Cry 23 protein, a Cry 36 protein, a Cry37 protein, a Cry34 protein together with a Cry35 protein, a binary insecticidal protein CryET33 and CryET34, a binary insecticidal protein TIC100 and TIC101, a binary insecticidal protein PS149B1, a VIP, a TIC900 or related protein, a TIC901, TIC1201, TIC407, TIC417, a modified Cry3A protein, or hybrid proteins or chimeras made from any of the preceding insecticidal proteins. The insecticidal protein may be any other insecticidal protein derived from B. thuringiensis known in the art to be insecticidal (see for example, Palma et al., 2014, Toxins 6: 3296-3325, and references within; Berry and Crickmore, 2017, J of Invertebrate Pathology 142: 16-22, and reference within).

In other embodiments, the transgenic plant may produce an interfering RNA of the invention and a second insecticidal agent which is derived from sources other than B. thuringiensis. The second insecticidal agent can be an agent selected from the group comprising a patatin, a protease, a protease inhibitor, a chitinase, a urease, an alpha-amylase inhibitor, a pore-forming protein, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, a Clostridium spp. (such as C. bifermentans) insecticidal protein, and a lignin. In other embodiments, the second agent may be at least one insecticidal protein derived from an insecticidal toxin complex (Tc) from Photorhabdus, Xenorhabus, Serratia, or Yersinia. In other embodiments, the insecticidal protein may be an ADP-ribosyltransferase derived from an insecticidal bacteria, such as Photorhabdus spp. In other embodiments, the insecticidal protein may be a VIP protein, such as VIP1 or VIP2 from B. cereus. In still other embodiments, the insecticidal protein may be a binary toxin derived from an insecticidal bacteria, such as ISP1A and ISP2A from B. laterosporous or BinA and BinB from L. sphaericus. In still other embodiments, the insecticidal protein may be engineered or may be a hybrid or chimera of any of the preceding insecticidal proteins.

In another embodiment, the transgenic plant and transgenic seed is a canola plant or canola seed. In another embodiment, the transgenic canola plant is provided by crossing a first transgenic canola plant comprising a dsRNA of the invention with a transgenic canola plant comprising a transgenic event, for example RoundupReady® Canola, Navigator™ Canola, Phytaseed™ Canola, or LibertyLink® Canola.

Even where the insecticide or insecticidal seed coating is active against a different insect, the insecticide or insecticidal seed coating is useful to expand the range of insect control, for example by adding an insecticide or insecticidal seed coating that has activity against lepidopteran insects to the transgenic plant or seed of the invention, which has activity against Coleopteran insects, the treated plant or coated transgenic seed controls both lepidopteran and Coleopteran insect pests.

In further embodiments, the invention encompasses a biological sample from a transgenic plant, seed, or parts thereof, of the invention, wherein the sample comprises a nucleic acid that is or encodes at least one strand of a dsRNA of the invention. In other embodiments, the invention encompasses a commodity product derived from a transgenic plant, seed, or parts thereof, of the invention. In some embodiments, the commodity product is selected from the group consisting of whole or processed seeds, beans, grains, kernels, hulls, meals, grits, flours, sugars, sugars, starches, protein concentrates, protein isolates, waxes, oils, extracts, juices, concentrates, liquids, syrups, feed, silage, fiber, paper or other food or product produced from plants. In some embodiments, the commodity product consists of whole seeds and comprises a nucleic acid that is or encodes at least one strand of a dsRNA of the invention. In some embodiments, the biological sample or commodity product is toxic to insects. In some embodiments, the transgenic plant is a transgenic canola plant.

The invention further encompasses a method of controlling a Coleopteran insect or a flea beetle comprising contacting the insect with a nucleic acid molecule that is or is capable of producing an interfering RNA molecule of the invention for inhibiting expression of a target gene in the insect thereby controlling the Coleopteran insect or the flea beetle. In some embodiments, the target gene comprises a coding sequence (i) having at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 1-102, or a complement thereof; (ii) comprising at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 1-102, or a complement thereof; (iii) comprising at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 1-102, or a complement thereof. In some embodiments the target gene coding sequence comprises SEQ ID NO: 1-102, or a complement thereof, or can hybridize under stringent conditions to a polynucleotide selected from the group consisting of SEQ ID NO: 1-102, and the complements thereof. In other embodiments, the interfering RNA molecule of the invention is complementary to a portion of a mRNA polynucleotide transcribable from the flea beetle target genes described herein.

In some embodiments of the method of controlling a Coleopteran insect pest or a flea beetle pest, the interfering RNA molecule of the invention comprises at least one dsRNA, wherein the dsRNA is a region of double-stranded RNA comprising annealed complementary strands, one strand of which comprises a sequence of at least 19 contiguous nucleotides which (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; or (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 409-612, or the complement thereof, or (iv) can hybridize under stringent conditions to a polynucleotide selected from the group consisting of SEQ ID NO: 409-612, and the complements thereof.

In some embodiments of the method of controlling a Coleopteran insect pest or a flea beetle pest, the interfering RNA molecule comprises, consists essentially of or consists of from 18, 19, 20 or 21 consecutive nucleotides to at least about 300 consecutive nucleotides of SEQ ID NO: 409-612. In other embodiments of the methods of the invention, the interfering RNA molecule of the invention comprises a dsRNA which comprises, consists essentially of or consists of any 21-mer subsequence of SEQ ID NO: 409-510 consisting of N to N+20 nucleotides, or any complement thereof. For example, an interfering RNA molecule of the invention comprises a dsRNA which comprises, consist essentially of or consists of any 21-mer subsequence of SEQ ID NO: 409, wherein N is nucleotide 1 to nucleotide 784 of SEQ ID NO: 409, or any complement thereof. In other words, the portion of the mRNA that is targeted comprises any of the 784 21 consecutive nucleotide subsequences i.e. 21-mers) of SEQ ID NO: 409, or any of their complementing sequences. It will be recognized that these 784 21 consecutive nucleotide subsequences include all possible 21 consecutive nucleotide subsequences from SEQ ID NO: 409 and from SEQ ID NO: 511, and their complements, as SEQ ID NO's 409 and 511 are all to the same target, namely Pa1, which also may be referred to as a P. armoraciae ortholog of Rpn12. It will similarly be recognized that all 21-mer subsequences of SEQ ID NO: 409-510 and all complement subsequences thereof, include all possible 21 consecutive nucleotide subsequences of SEQ ID NO: 511-612, and the complement subsequences thereof.

Similarly, in some embodiments of the method of controlling a Coleopteran insect pest or a flea beetle pest, an interfering RNA molecule of the invention comprises a dsRNA which comprises, consist essentially of or consists of any 21-mer subsequence of SEQ ID NO: 410, wherein N is nucleotide 1 to nucleotide 436 of SEQ ID NO: 410, or any complement thereof. Another interfering RNA molecule of the invention comprises a dsRNA which comprises, consist essentially of or consists of any 21-mer subsequence of SEQ ID NO: 411, wherein N is nucleotide 1 to nucleotide 752 of SEQ ID NO: 411, or any complement thereof. Another interfering RNA molecule of the invention comprises a dsRNA which comprises, consist essentially of or consists of any 21-mer subsequence of SEQ ID NO: 412, wherein N is nucleotide 1 to nucleotide 811 of SEQ ID NO: 412, or any complement thereof. Another interfering RNA molecule of the invention comprises a dsRNA which comprises, consist essentially of or consists of any 21-mer subsequence of SEQ ID NO: 413, wherein N is nucleotide 1 to nucleotide 556 of SEQ ID NO: 413, or any complement thereof. Another interfering RNA molecule of the invention comprises a dsRNA which comprises, consist essentially of or consists of any 21-mer subsequence of SEQ ID NO: 414, wherein N is nucleotide 1 to nucleotide 442 of SEQ ID NO: 414, or any complement thereof. Another interfering RNA molecule of the invention comprises a dsRNA which comprises, consist essentially of or consists of any 21-mer subsequence of SEQ ID NO: 415, wherein N is nucleotide 1 to nucleotide 850 of SEQ ID NO: 415, or any complement thereof. Another interfering RNA molecule of the invention comprises a dsRNA which comprises, consist essentially of or consists of any 21-mer subsequence of SEQ ID NO: 416, wherein N is nucleotide 1 to nucleotide 352 of SEQ ID NO: 416, or any complement thereof. Another interfering RNA molecule of the invention comprises a dsRNA which comprises, consist essentially of or consists of any 21-mer subsequence of SEQ ID NO: 417, wherein N is nucleotide 1 to nucleotide 334 of SEQ ID NO: 417, or any complement thereof. Another interfering RNA molecule of the invention comprises a dsRNA which comprises, consist essentially of or consists of any 21-mer subsequence of SEQ ID NO: 418, wherein N is nucleotide 1 to nucleotide 355 of SEQ ID NO: 418, or any complement thereof. Another interfering RNA molecule of the invention comprises a dsRNA which comprises, consist essentially of or consists of any 21-mer subsequence of SEQ ID NO: 419, wherein N is nucleotide 1 to nucleotide 5674 of SEQ ID NO: 419, or any complement thereof. Another interfering RNA molecule of the invention comprises a dsRNA which comprises, consist essentially of or consists of any 21-mer subsequence of SEQ ID NO: 420, wherein N is nucleotide 1 to nucleotide 511 of SEQ ID NO: 420, or any complement thereof. Another interfering RNA molecule of the invention comprises a dsRNA which comprises, consist essentially of or consists of any 21-mer subsequence of SEQ ID NO: 421, wherein N is nucleotide 1 to nucleotide 388 of SEQ ID NO: 421, or any complement thereof. Another interfering RNA molecule of the invention comprises a dsRNA which comprises, consist essentially of or consists of any 21-mer subsequence of SEQ ID NO: 422, wherein N is nucleotide 1 to nucleotide 385 of SEQ ID NO: 422, or any complement thereof. Another interfering RNA molecule of the invention comprises a dsRNA which comprises, consist essentially of or consists of any 21-mer subsequence of SEQ ID NO: 423, wherein N is nucleotide 1 to nucleotide 400 of SEQ ID NO: 423, or any complement thereof. Another interfering RNA molecule of the invention comprises a dsRNA which comprises, consist essentially of or consists of any 21-mer subsequence of SEQ ID NO: 424, wherein N is nucleotide 1 to nucleotide 1702 of SEQ ID NO: 424, or any complement thereof. Another interfering RNA molecule of the invention comprises a dsRNA which comprises, consist essentially of or consists of any 21-mer subsequence of SEQ ID NO: 425, wherein N is nucleotide 1 to nucleotide 3973 of SEQ ID NO: 425, or any complement thereof. Another interfering RNA molecule of the invention comprises a dsRNA which comprises, consist essentially of or consists of any 21-mer subsequence of SEQ ID NO: 426, wherein N is nucleotide 1 to nucleotide 784 of SEQ ID NO: 426, or any complement thereof. Another interfering RNA molecule of the invention comprises a dsRNA which comprises, consist essentially of or consists of any 21-mer subsequence of SEQ ID NO: 427, wherein N is nucleotide 1 to nucleotide 436 of SEQ ID NO: 427, or any complement thereof. Another interfering RNA molecule of the invention comprises a dsRNA which comprises, consist essentially of or consists of any 21-mer subsequence of SEQ ID NO: 428, wherein N is nucleotide 1 to nucleotide 787 of SEQ ID NO: 428, or any complement thereof. Another interfering RNA molecule of the invention comprises a dsRNA which comprises, consist essentially of or consists of any 21-mer subsequence of SEQ ID NO: 429, wherein N is nucleotide 1 to nucleotide 805 of SEQ ID NO: 429, or any complement thereof. Another interfering RNA molecule of the invention comprises a dsRNA which comprises, consist essentially of or consists of any 21-mer subsequence of SEQ ID NO: 430, wherein N is nucleotide 1 to nucleotide 562 of SEQ ID NO: 430, or any complement thereof. Another interfering RNA molecule of the invention comprises a dsRNA which comprises, consist essentially of or consists of any 21-mer subsequence of SEQ ID NO: 431, wherein N is nucleotide 1 to nucleotide 439 of SEQ ID NO: 431, or any complement thereof. Another interfering RNA molecule of the invention comprises a dsRNA which comprises, consist essentially of or consists of any 21-mer subsequence of SEQ ID NO: 432, wherein N is nucleotide 1 to nucleotide 856 of SEQ ID NO: 432, or any complement thereof. Another interfering RNA molecule of the invention comprises a dsRNA which comprises, consist essentially of or consists of any 21-mer subsequence of SEQ ID NO: 433, wherein N is nucleotide 1 to nucleotide 352 of SEQ ID NO: 433, or any complement thereof. Another interfering RNA molecule of the invention comprises a dsRNA which comprises, consist essentially of or consists of any 21-mer subsequence of SEQ ID NO: 434, wherein N is nucleotide 1 to nucleotide 334 of SEQ ID NO: 434, or any complement thereof. Another interfering RNA molecule of the invention comprises a dsRNA which comprises, consist essentially of or consists of any 21-mer subsequence of SEQ ID NO: 435, wherein N is nucleotide 1 to nucleotide 355 of SEQ ID NO: 435, or any complement thereof. Another interfering RNA molecule of the invention comprises a dsRNA which comprises, consist essentially of or consists of any 21-mer subsequence of SEQ ID NO: 436, wherein N is nucleotide 1 to nucleotide 5674 of SEQ ID NO: 436, or any complement thereof. Another interfering RNA molecule of the invention comprises a dsRNA which comprises, consist essentially of or consists of any 21-mer subsequence of SEQ ID NO: 437, wherein N is nucleotide 1 to nucleotide 511 of SEQ ID NO: 437, or any complement thereof. Another interfering RNA molecule of the invention comprises a dsRNA which comprises, consist essentially of or consists of any 21-mer subsequence of SEQ ID NO: 438, wherein N is nucleotide 1 to nucleotide 388 of SEQ ID NO: 438, or any complement thereof. Another interfering RNA molecule of the invention comprises a dsRNA which comprises, consist essentially of or consists of any 21-mer subsequence of SEQ ID NO: 439, wherein N is nucleotide 1 to nucleotide 385 of SEQ ID NO: 439, or any complement thereof. Another interfering RNA molecule of the invention comprises a dsRNA which comprises, consist essentially of or consists of any 21-mer subsequence of SEQ ID NO: 440, wherein N is nucleotide 1 to nucleotide 400 of SEQ ID NO: 440, or any complement thereof. Another interfering RNA molecule of the invention comprises a dsRNA which comprises, consist essentially of or consists of any 21-mer subsequence of SEQ ID NO: 441, wherein N is nucleotide 1 to nucleotide 2047 of SEQ ID NO: 441, or any complement thereof. Another interfering RNA molecule of the invention comprises a dsRNA which comprises, consist essentially of or consists of any 21-mer subsequence of SEQ ID NO: 442, wherein N is nucleotide 1 to nucleotide 3964 of SEQ ID NO: 442, or any complement thereof. Another interfering RNA molecule of the invention comprises a dsRNA which comprises, consist essentially of or consists of any 21-mer subsequence of SEQ ID NO: 443, wherein N is nucleotide 1 to nucleotide 784 of SEQ ID NO: 443, or any complement thereof. Another interfering RNA molecule of the invention comprises a dsRNA which comprises, consist essentially of or consists of any 21-mer subsequence of SEQ ID NO: 444, wherein N is nucleotide 1 to nucleotide 436 of SEQ ID NO: 444, or any complement thereof. Another interfering RNA molecule of the invention comprises a dsRNA which comprises, consist essentially of or consists of any 21-mer subsequence of SEQ ID NO: 445, wherein N is nucleotide 1 to nucleotide 787 of SEQ ID NO: 445, or any complement thereof. Another interfering RNA molecule of the invention comprises a dsRNA which comprises, consist essentially of or consists of any 21-mer subsequence of SEQ ID NO: 446, wherein N is nucleotide 1 to nucleotide 811 of SEQ ID NO: 446, or any complement thereof. Another interfering RNA molecule of the invention comprises a dsRNA which comprises, consist essentially of or consists of any 21-mer subsequence of SEQ ID NO: 447, wherein N is nucleotide 1 to nucleotide 487 of SEQ ID NO: 447, or any complement thereof. Another interfering RNA molecule of the invention comprises a dsRNA which comprises, consist essentially of or consists of any 21-mer subsequence of SEQ ID NO: 448, wherein N is nucleotide 1 to nucleotide 406 of SEQ ID NO: 448, or any complement thereof. Another interfering RNA molecule of the invention comprises a dsRNA which comprises, consist essentially of or consists of any 21-mer subsequence of SEQ ID NO: 449, wherein N is nucleotide 1 to nucleotide 856 of SEQ ID NO: 449, or any complement thereof. Another interfering RNA molecule of the invention comprises a dsRNA which comprises, consist essentially of or consists of any 21-mer subsequence of SEQ ID NO: 450, wherein N is nucleotide 1 to nucleotide 251 of SEQ ID NO: 450, or any complement thereof. Another interfering RNA molecule of the invention comprises a dsRNA which comprises, consist essentially of or consists of any 21-mer subsequence of SEQ ID NO: 451, wherein N is nucleotide 1 to nucleotide 334 of SEQ ID NO: 451, or any complement thereof. Another interfering RNA molecule of the invention comprises a dsRNA which comprises, consist essentially of or consists of any 21-mer subsequence of SEQ ID NO: 452, wherein N is nucleotide 1 to nucleotide 355 of SEQ ID NO: 452, or any complement thereof. Another interfering RNA molecule of the invention comprises a dsRNA which comprises, consist essentially of or consists of any 21-mer subsequence of SEQ ID NO: 453, wherein N is nucleotide 1 to nucleotide 5674 of SEQ ID NO: 453, or any complement thereof. Another interfering RNA molecule of the invention comprises a dsRNA which comprises, consist essentially of or consists of any 21-mer subsequence of SEQ ID NO: 454, wherein N is nucleotide 1 to nucleotide 511 of SEQ ID NO: 454, or any complement thereof. Another interfering RNA molecule of the invention comprises a dsRNA which comprises, consist essentially of or consists of any 21-mer subsequence of SEQ ID NO: 455, wherein N is nucleotide 1 to nucleotide 388 of SEQ ID NO: 455, or any complement thereof. Another interfering RNA molecule of the invention comprises a dsRNA which comprises, consist essentially of or consists of any 21-mer subsequence of SEQ ID NO: 456, wherein N is nucleotide 1 to nucleotide 385 of SEQ ID NO: 456, or any complement thereof. Another interfering RNA molecule of the invention comprises a dsRNA which comprises, consist essentially of or consists of any 21-mer subsequence of SEQ ID NO: 457, wherein N is nucleotide 1 to nucleotide 400 of SEQ ID NO: 457, or any complement thereof. Another interfering RNA molecule of the invention comprises a dsRNA which comprises, consist essentially of or consists of any 21-mer subsequence of SEQ ID NO: 458, wherein N is nucleotide 1 to nucleotide 1189 of SEQ ID NO: 458, or any complement thereof. Another interfering RNA molecule of the invention comprises a dsRNA which comprises, consist essentially of or consists of any 21-mer subsequence of SEQ ID NO: 459, wherein N is nucleotide 1 to nucleotide 3514 of SEQ ID NO: 459, or any complement thereof. Another interfering RNA molecule of the invention comprises a dsRNA which comprises, consist essentially of or consists of any 21-mer subsequence of SEQ ID NO: 460, wherein N is nucleotide 1 to nucleotide 784 of SEQ ID NO: 460, or any complement thereof. Another interfering RNA molecule of the invention comprises a dsRNA which comprises, consist essentially of or consists of any 21-mer subsequence of SEQ ID NO: 461, wherein N is nucleotide 1 to nucleotide 436 of SEQ ID NO: 461, or any complement thereof. Another interfering RNA molecule of the invention comprises a dsRNA which comprises, consist essentially of or consists of any 21-mer subsequence of SEQ ID NO: 462, wherein N is nucleotide 1 to nucleotide 787 of SEQ ID NO: 462, or any complement thereof. Another interfering RNA molecule of the invention comprises a dsRNA which comprises, consist essentially of or consists of any 21-mer subsequence of SEQ ID NO: 463, wherein N is nucleotide 1 to nucleotide 811 of SEQ ID NO: 463, or any complement thereof. Another interfering RNA molecule of the invention comprises a dsRNA which comprises, consist essentially of or consists of any 21-mer subsequence of SEQ ID NO: 464, wherein N is nucleotide 1 to nucleotide 556 of SEQ ID NO: 464, or any complement thereof. Another interfering RNA molecule of the invention comprises a dsRNA which comprises, consist essentially of or consists of any 21-mer subsequence of SEQ ID NO: 465, wherein N is nucleotide 1 to nucleotide 439 of SEQ ID NO: 465, or any complement thereof. Another interfering RNA molecule of the invention comprises a dsRNA which comprises, consist essentially of or consists of any 21-mer subsequence of SEQ ID NO: 466, wherein N is nucleotide 1 to nucleotide 856 of SEQ ID NO: 466, or any complement thereof. Another interfering RNA molecule of the invention comprises a dsRNA which comprises, consist essentially of or consists of any 21-mer subsequence of SEQ ID NO: 467, wherein N is nucleotide 1 to nucleotide 352 of SEQ ID NO: 467, or any complement thereof. Another interfering RNA molecule of the invention comprises a dsRNA which comprises, consist essentially of or consists of any 21-mer subsequence of SEQ ID NO: 468, wherein N is nucleotide 1 to nucleotide 334 of SEQ ID NO: 468, or any complement thereof. Another interfering RNA molecule of the invention comprises a dsRNA which comprises, consist essentially of or consists of any 21-mer subsequence of SEQ ID NO: 469, wherein N is nucleotide 1 to nucleotide 355 of SEQ ID NO: 469, or any complement thereof. Another interfering RNA molecule of the invention comprises a dsRNA which comprises, consist essentially of or consists of any 21-mer subsequence of SEQ ID NO: 470, wherein N is nucleotide 1 to nucleotide 5260 of SEQ ID NO: 470, or any complement thereof. Another interfering RNA molecule of the invention comprises a dsRNA which comprises, consist essentially of or consists of any 21-mer subsequence of SEQ ID NO: 471, wherein N is nucleotide 1 to nucleotide 511 of SEQ ID NO: 471, or any complement thereof. Another interfering RNA molecule of the invention comprises a dsRNA which comprises, consist essentially of or consists of any 21-mer subsequence of SEQ ID NO: 472, wherein N is nucleotide 1 to nucleotide 388 of SEQ ID NO: 472, or any complement thereof. Another interfering RNA molecule of the invention comprises a dsRNA which comprises, consist essentially of or consists of any 21-mer subsequence of SEQ ID NO: 473, wherein N is nucleotide 1 to nucleotide 385 of SEQ ID NO: 473, or any complement thereof. Another interfering RNA molecule of the invention comprises a dsRNA which comprises, consist essentially of or consists of any 21-mer subsequence of SEQ ID NO: 474, wherein N is nucleotide 1 to nucleotide 400 of SEQ ID NO: 474, or any complement thereof. Another interfering RNA molecule of the invention comprises a dsRNA which comprises, consist essentially of or consists of any 21-mer subsequence of SEQ ID NO: 475, wherein N is nucleotide 1 to nucleotide 2044 of SEQ ID NO: 475, or any complement thereof. Another interfering RNA molecule of the invention comprises a dsRNA which comprises, consist essentially of or consists of any 21-mer subsequence of SEQ ID NO: 476, wherein N is nucleotide 1 to nucleotide 3511 of SEQ ID NO: 476, or any complement thereof. Another interfering RNA molecule of the invention comprises a dsRNA which comprises, consist essentially of or consists of any 21-mer subsequence of SEQ ID NO: 477, wherein N is nucleotide 1 to nucleotide 784 of SEQ ID NO: 477, or any complement thereof. Another interfering RNA molecule of the invention comprises a dsRNA which comprises, consist essentially of or consists of any 21-mer subsequence of SEQ ID NO: 478, wherein N is nucleotide 1 to nucleotide 412 of SEQ ID NO: 478, or any complement thereof. Another interfering RNA molecule of the invention comprises a dsRNA which comprises, consist essentially of or consists of any 21-mer subsequence of SEQ ID NO: 479, wherein N is nucleotide 1 to nucleotide 676 of SEQ ID NO: 479, or any complement thereof. Another interfering RNA molecule of the invention comprises a dsRNA which comprises, consist essentially of or consists of any 21-mer subsequence of SEQ ID NO: 480, wherein N is nucleotide 1 to nucleotide 811 of SEQ ID NO: 480, or any complement thereof. Another interfering RNA molecule of the invention comprises a dsRNA which comprises, consist essentially of or consists of any 21-mer subsequence of SEQ ID NO: 481, wherein N is nucleotide 1 to nucleotide 556 of SEQ ID NO: 481, or any complement thereof. Another interfering RNA molecule of the invention comprises a dsRNA which comprises, consist essentially of or consists of any 21-mer subsequence of SEQ ID NO: 482, wherein N is nucleotide 1 to nucleotide 439 of SEQ ID NO: 482, or any complement thereof. Another interfering RNA molecule of the invention comprises a dsRNA which comprises, consist essentially of or consists of any 21-mer subsequence of SEQ ID NO: 483, wherein N is nucleotide 1 to nucleotide 856 of SEQ ID NO: 483, or any complement thereof. Another interfering RNA molecule of the invention comprises a dsRNA which comprises, consist essentially of or consists of any 21-mer subsequence of SEQ ID NO: 484, wherein N is nucleotide 1 to nucleotide 352 of SEQ ID NO: 484, or any complement thereof. Another interfering RNA molecule of the invention comprises a dsRNA which comprises, consist essentially of or consists of any 21-mer subsequence of SEQ ID NO: 485, wherein N is nucleotide 1 to nucleotide 334 of SEQ ID NO: 485, or any complement thereof. Another interfering RNA molecule of the invention comprises a dsRNA which comprises, consist essentially of or consists of any 21-mer subsequence of SEQ ID NO: 486, wherein N is nucleotide 1 to nucleotide 361 of SEQ ID NO: 486, or any complement thereof. Another interfering RNA molecule of the invention comprises a dsRNA which comprises, consist essentially of or consists of any 21-mer subsequence of SEQ ID NO: 487, wherein N is nucleotide 1 to nucleotide 5668 of SEQ ID NO: 487, or any complement thereof. Another interfering RNA molecule of the invention comprises a dsRNA which comprises, consist essentially of or consists of any 21-mer subsequence of SEQ ID NO: 488, wherein N is nucleotide 1 to nucleotide 457 of SEQ ID NO: 488, or any complement thereof. Another interfering RNA molecule of the invention comprises a dsRNA which comprises, consist essentially of or consists of any 21-mer subsequence of SEQ ID NO: 489, wherein N is nucleotide 1 to nucleotide 388 of SEQ ID NO: 489, or any complement thereof. Another interfering RNA molecule of the invention comprises a dsRNA which comprises, consist essentially of or consists of any 21-mer subsequence of SEQ ID NO: 490, wherein N is nucleotide 1 to nucleotide 385 of SEQ ID NO: 490, or any complement thereof. Another interfering RNA molecule of the invention comprises a dsRNA which comprises, consist essentially of or consists of any 21-mer subsequence of SEQ ID NO: 491, wherein N is nucleotide 1 to nucleotide 400 of SEQ ID NO: 491, or any complement thereof. Another interfering RNA molecule of the invention comprises a dsRNA which comprises, consist essentially of or consists of any 21-mer subsequence of SEQ ID NO: 492, wherein N is nucleotide 1 to nucleotide 2044 of SEQ ID NO: 492, or any complement thereof. Another interfering RNA molecule of the invention comprises a dsRNA which comprises, consist essentially of or consists of any 21-mer subsequence of SEQ ID NO: 493, wherein N is nucleotide 1 to nucleotide 3970 of SEQ ID NO: 493, or any complement thereof. Another interfering RNA molecule of the invention comprises a dsRNA which comprises, consist essentially of or consists of any 21-mer subsequence of SEQ ID NO: 494, wherein N is nucleotide 1 to nucleotide 784 of SEQ ID NO: 494, or any complement thereof. Another interfering RNA molecule of the invention comprises a dsRNA which comprises, consist essentially of or consists of any 21-mer subsequence of SEQ ID NO: 495, wherein N is nucleotide 1 to nucleotide 436 of SEQ ID NO: 495, or any complement thereof. Another interfering RNA molecule of the invention comprises a dsRNA which comprises, consist essentially of or consists of any 21-mer subsequence of SEQ ID NO: 496, wherein N is nucleotide 1 to nucleotide 787 of SEQ ID NO: 496, or any complement thereof. Another interfering RNA molecule of the invention comprises a dsRNA which comprises, consist essentially of or consists of any 21-mer subsequence of SEQ ID NO: 497, wherein N is nucleotide 1 to nucleotide 811 of SEQ ID NO: 497, or any complement thereof. Another interfering RNA molecule of the invention comprises a dsRNA which comprises, consist essentially of or consists of any 21-mer subsequence of SEQ ID NO: 498, wherein N is nucleotide 1 to nucleotide 487 of SEQ ID NO: 498, or any complement thereof. Another interfering RNA molecule of the invention comprises a dsRNA which comprises, consist essentially of or consists of any 21-mer subsequence of SEQ ID NO: 499, wherein N is nucleotide 1 to nucleotide 439 of SEQ ID NO: 499, or any complement thereof. Another interfering RNA molecule of the invention comprises a dsRNA which comprises, consist essentially of or consists of any 21-mer subsequence of SEQ ID NO: 500, wherein N is nucleotide 1 to nucleotide 856 of SEQ ID NO: 500, or any complement thereof. Another interfering RNA molecule of the invention comprises a dsRNA which comprises, consist essentially of or consists of any 21-mer subsequence of SEQ ID NO: 501, wherein N is nucleotide 1 to nucleotide 352 of SEQ ID NO: 501, or any complement thereof. Another interfering RNA molecule of the invention comprises a dsRNA which comprises, consist essentially of or consists of any 21-mer subsequence of SEQ ID NO: 502, wherein N is nucleotide 1 to nucleotide 334 of SEQ ID NO: 502, or any complement thereof. Another interfering RNA molecule of the invention comprises a dsRNA which comprises, consist essentially of or consists of any 21-mer subsequence of SEQ ID NO: 503, wherein N is nucleotide 1 to nucleotide 355 of SEQ ID NO: 503, or any complement thereof. Another interfering RNA molecule of the invention comprises a dsRNA which comprises, consist essentially of or consists of any 21-mer subsequence of SEQ ID NO: 504, wherein N is nucleotide 1 to nucleotide 4528 of SEQ ID NO: 504, or any complement thereof. Another interfering RNA molecule of the invention comprises a dsRNA which comprises, consist essentially of or consists of any 21-mer subsequence of SEQ ID NO: 505, wherein N is nucleotide 1 to nucleotide 511 of SEQ ID NO: 505, or any complement thereof. Another interfering RNA molecule of the invention comprises a dsRNA which comprises, consist essentially of or consists of any 21-mer subsequence of SEQ ID NO: 506, wherein N is nucleotide 1 to nucleotide 388 of SEQ ID NO: 506, or any complement thereof. Another interfering RNA molecule of the invention comprises a dsRNA which comprises, consist essentially of or consists of any 21-mer subsequence of SEQ ID NO: 507, wherein N is nucleotide 1 to nucleotide 385 of SEQ ID NO: 507, or any complement thereof. Another interfering RNA molecule of the invention comprises a dsRNA which comprises, consist essentially of or consists of any 21-mer subsequence of SEQ ID NO: 508, wherein N is nucleotide 1 to nucleotide 400 of SEQ ID NO: 508, or any complement thereof. Another interfering RNA molecule of the invention comprises a dsRNA which comprises, consist essentially of or consists of any 21-mer subsequence of SEQ ID NO: 509, wherein N is nucleotide 1 to nucleotide 1939 of SEQ ID NO: 509, or any complement thereof. Another interfering RNA molecule of the invention comprises a dsRNA which comprises, consist essentially of or consists of any 21-mer subsequence of SEQ ID NO: 510, wherein N is nucleotide 1 to nucleotide 3970 of SEQ ID NO: 510, or any complement thereof.

In some embodiments of the method of controlling a flea beetle insect pest, the insect is from the tribe Alticini. In further embodiments, the insect is a species of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In embodiments, the insect is a species selected from the group consisting of Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).

In other embodiments of the method of controlling a Coleopteran insect pest or a flea beetle pest, the contacting comprises (a) planting a transgenic seed capable of producing a transgenic plant that expresses the nucleic acid molecule, wherein the insect feeds on the transgenic plant, or part thereof; or (b) applying a composition comprising the nucleic acid molecule to a seed or plant, or part thereof, wherein the insect feeds on the seed, the plant, or a part thereof. In some embodiments, the transgenic seed and the transgenic plant is a canola seed or a canola plant. In other embodiments the seed or plant is a canola seed or a canola plant.

The invention also encompasses a method of controlling a flea beetle comprising contacting the flea beetle with a nucleic acid molecule that is or is capable of producing the interfering RNA molecule of the invention for inhibiting expression of a target gene in the flea beetle, and also contacting the flea beetle with at least a second insecticidal agent for controlling the flea beetle, wherein said second insecticidal agent comprises a B. thuringiensis insecticidal protein, thereby controlling the flea beetle. The invention also encompasses a method for controlling flea beetle pests on a plant, comprising topically applying to said plant a pesticide composition comprising an interfering RNA of the invention and at least a second insecticidal agent for controlling the flea beetle, wherein said second insecticidal agent does not comprise a B. thuringiensis insecticidal protein, and providing said plant in the diet of said flea beetle. The invention also encompasses a method wherein the second insecticidal agent comprises a patatin, a protease, a protease inhibitor, a urease, an alpha-amylase inhibitor, a pore-forming protein, a lectin, an engineered antibody or antibody fragment, or a chitinase. The second insecticidal agent may also be a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium ssp. insecticidal protein, a Yersinia entomophaga insecticidal protein, a Paenibacillus popiliae insecticidal protein, or a Clostridium spp. insecticidal protein.

The invention also encompasses a method of reducing an adult Coleopteran insect population or an adult flea beetle population on a transgenic plant expressing a Cry protein, a hybrid Cry protein or modified Cry protein comprising expressing in the transgenic plant a nucleic acid molecule that is or is capable of producing an interfering RNA molecule of the invention capable of inhibiting expression of a target gene as described herein in an adult insect, thereby reducing the adult Coleopteran insect population or adult flea beetle population.

In some embodiments, the invention encompasses a method of reducing the level of a target mRNA transcribable from a target gene as described herein in a Coleopteran insect or a flea beetle comprising contacting the insect with a composition comprising the interfering RNA molecule of the invention, wherein the interfering RNA molecule reduces the level of the target mRNA in a cell of the insect. In some embodiments, the interfering RNA of the method comprises at least one dsRNA, wherein the dsRNA is a region of double-stranded RNA comprising annealed complementary strands, one strand of which comprises a sequence of at least 19 contiguous nucleotides which (i) has at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, to at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (ii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of SEQ ID NO: 409-612, or the complement thereof; (iii) comprises at least a 19, at least a 20, at least a 21, at least a 22, at least a 23, at least a 24, at least a 25, at least a 26, at least a 27, at least a 28, at least a 29, at least a 30, at least a 35, at least a 40, at least a 45, at least a 50, at least a 55, at least a 60, at least a a 65, at least a 70, at least a 75, at least a 80, at least a 85, at least a 90, at least a 95, at least a 100, at least a 110, at least a 120, at least a 130, at least a 140, at least a 150, at least a 160, at least a 170, at least a 180, at least a 190, at least a 200, at least a 210, at least a 220, at least a 230, at least a 240, at least a 250, at least a 260, at least a 270, at least a 280, at least a 290, or at least a 300 contiguous nucleotide fragment of a nucleotide sequence encoding an amino acid sequence encoded by SEQ ID NO: 409-612, or the complement thereof, or (iv) can hybridize under stringent conditions to a polynucleotide selected from the group consisting of SEQ ID NO: 409-612, and the complements thereof, wherein the interfering RNA molecule has insecticidal activity against the target Coleopteran insect or a flea beetle. In another embodiment, the contacting is achieved by the target insect feeding on the composition. In other embodiments, production of the protein encoded by the target mRNA is reduced. In other embodiments, the target protein comprises an amino acid having at least about 80%, at least about 85%, at least about 90%, at least about 91%, at least about 92%, at least about 93%, at least about 94%, at least about 95%, at least about 96%, at least about 97%, at least about 98% or at least about 99% identity to SEQ ID NO: 613-714. In other embodiments the target protein comprises SEQ ID NO: 613-714. In other embodiments, the interfering RNA is contacted with a Coleopteran insect or a flea beetle through a transgenic organism expressing the interfering RNA. In other embodiments, the transgenic organism is a transgenic plant, a transgenic microorganism, a transgenic bacterium or a transgenic endophyte. In other embodiments, the interfering RNA is contacted with a Coleopteran insect or a flea beetle by topically applying an interfering RNA in an acceptable agricultural carrier to a plant or plant part on which the insect feeds. In some embodiments, the interfering RNA that reduces the level of a target mRNA transcribable from a target gene described herein is lethal to the Coleopteran insect or flea beetle. In some embodiments, the flea beetle a member of the Alticini tribe. In some embodiments, the flea beetle is a species of a genus selected from the group consisting of the genera Altica, Anthobiodes, Aphthona, Aphthonaltica, Aphthonoides, Apteopeda, Argopistes, Argopus, Arrhenocoela, Batophila, Blepharida, Chaetocnema, Clitea, Crepidodera, Derocrepis, Dibolia, Disonycha, Epitrix, Hermipyxis, Hermaeophaga, Hespera, Hippuriphila, Horaia, Hyphasis, Lipromima, Liprus, Longitarsus, Luperomorpha, Lythraria, Manobia, Mantura, Meishania, Minota, Mniophila, Neicrepidodera, Nonarthra, Novofoudrasia, Ochrosis, Oedionychis, Oglobinia, Omeisphaera, Ophrida, Orestia, Paragopus, Pentamesa, Philopona, Phygasia, Phyllotreta, Podagrica, Podagricomela, Podontia, Pseudodera, Psylliodes, Sangariola, Sinaltica, Sphaeroderma, Systena, Trachyaphthona, Xuthea, and Zipangia. In embodiments, the insect is a species selected from the group consisting of Altica ambiens (alder flea beetle), Altica canadensis (prairie flea beetle), Altica chalybaea (grape flea beetle), Altica prasina (poplar flea beetle), Altica rosae (rose flea beetle), Altica sylvia (blueberry flea beetle), Altica ulmi (elm flea beetle), Chaetocnema pulicaria (corn flea beetle), Chaetocnema conofinis (sweet potato flea beetle), Epitrix cucumeris (potato flea beetle), Systena blanda (palestripped flea beetle), and Systena frontalis (redheaded flea beetle). In embodiments, the insect is a species selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).

In some embodiments, the invention encompasses a method of conferring Coleopteran insect tolerance or flea beetle tolerance to a plant, or part thereof, comprising introducing into the plant, or part thereof, an interfering RNA molecule, a dsRNA molecule, a nucleic acid construct, a chimeric nucleic acid molecule, an artificial plant microRNA precursor molecule and/or a composition of the invention, wherein the dsRNA molecule, nucleic acid construct, chimeric nucleic acid molecule, artificial plant microRNA precursor molecule and/or composition of the invention are toxic to the insect, thereby conferring tolerance of the plant or part thereof to the Coleopteran insect or flea beetle. In other embodiments, the introducing step is performed by transforming a plant cell and producing the transgenic plant from the transformed plant cell. In still other embodiments, the introducing step is performed by breeding two plants together.

In other embodiments, the invention encompasses a method of reducing damage to the plant fed upon by a flea beetle, comprising introducing into cells of the plant an interfering RNA molecule, a dsRNA, a nucleic acid molecule, a nucleic acid construct, a chimeric nucleic acid molecule, an artificial plant microRNA precursor molecule and/or a composition of the invention, wherein the dsRNA, nucleic acid molecule, nucleic acid construct, chimeric nucleic acid molecule, artificial plant microRNA precursor molecule and/or composition of the invention are toxic to the flea beetle, thereby reducing damage to the plant. In other embodiments, the introducing step is performed by transforming a plant cell and producing the transgenic plant from the transformed plant cell. In still other embodiments, the introducing step is performed by breeding two plants together.

In still other embodiments, the invention encompasses a method of producing a transgenic plant cell having toxicity to a Coleopteran insect or flea beetle, comprising introducing into a plant cell an interfering RNA molecule, a dsRNA, a nucleic acid molecule, a nucleic acid construct, a chimeric nucleic acid molecule, an artificial plant microRNA precursor molecule and/or a composition of the invention, thereby producing the transgenic plant cell having toxicity to the insect compared to a control plant cell. In some embodiments, the invention encompasses a plurality of transgenic plant cells produced by this method. In other embodiments, the plurality of transgenic plant cells is grown under conditions which include natural sunlight. In other embodiments, the introducing step is performed by transforming a plant cell and producing the transgenic plant from the transformed plant cell. In still other embodiments, the introducing step is performed by breeding two plants together.

In some embodiments, the invention encompasses a method of producing a transgenic plant having enhanced tolerance to Coleopteran or flea beetle feeding damage, comprising introducing into a plant an interfering RNA molecule, a dsRNA, a nucleic acid molecule, a nucleic acid construct, a chimeric nucleic acid molecule, an artificial plant microRNA precursor molecule and/or a composition of the invention, thereby producing a transgenic plant having enhanced tolerance to Coleopteran or flea beetle feeding damage compared to a control plant. In other embodiments, the introducing step is performed by transforming a plant cell and producing the transgenic plant from the transformed plant cell. In still other embodiments, the introducing step is performed by breeding two plants together.

In some embodiments, the invention encompasses a method of providing a canola grower with a means of controlling a Coleopteran insect pest population or a flea beetle pest population in a canola crop comprising (a) selling or providing to the grower transgenic canola seed that comprises an interfering RNA, a nucleic acid molecule, a nucleic acid construct, a chimeric nucleic acid molecule, an artificial plant microRNA precursor molecule and/or a composition of the invention; and (b) advertising to the grower that the transgenic canola seed produce transgenic canola plants that control a Coleopteran or flea beetle pest population.

In some embodiments, the invention encompasses a method of identifying a target gene for using as a RNAi strategy for the control of a plant pest for RNAi in a Coleopteran plant pest, said method comprising the steps of a) producing a primer pair which can amplify a sequence that is or is orthologous to SEQ ID NO: 1-204, or a complement thereof; b) amplifying an orthologous target from a nucleic acid sample of the plant pest; c) identifying a sequence of an orthologous target gene; d) producing an interfering RNA molecule, wherein the RNA comprises at least one dsRNA, wherein the dsRNA is a region of double-stranded RNA comprising annealed complementary strands, one strand of which comprises a sequence of at least 19 contiguous nucleotides which is at least partially complementary to a target nucleotide sequence within a Coleopteran target gene, is obtained; and e) determining if the interfering RNA molecule has insecticidal activity on the plant pest. If the interfering RNA has insecticidal activity on the Coleopteran pest, a target gene for using in the control of the plant pest has been identified. In some embodiments, the plant pest is a Coleopteran plant pest.

### EXAMPLES

The invention will be further described by reference to the following detailed examples. These examples are provided for the purposes of illustration only, and are not intended to be limiting unless otherwise specified.

### Example 1: Identification of Potential RNAi Gene Targets in Flea Beetle Species

This example describes the cloning and sequencing of RNAi target genes and coding sequences from Psylliodes chrysocephala, Phyllotreta nemorum, Phyllotreta striolata, Phyllotreta cruciferae, and from Phyllotreta armoraciae insects.

Target gene selection was based on known lethal genes in other organisms, which were identified based on published disclosures including WO2012/143543, WO2012/143542, WO2018/026770, WO2018/026773, and WO2018/026774. From this analysis, several targets were identified. Each of these target genes is known to possess an allele(s) which is lethal, or is known to result in lethality when targeted by RNAi, in either Diabrotica virgifera virgifera, Leptinotarsa decemlineata, Lygus hesperus, or a combination thereof. Therefore, each of these targets were considered likely to confer an insecticidal effect when targeted with a dsRNA molecule against the native gene.

dsRNAs based on the selected targets were produced on an 96 well semi-automated library synthesis platform. Templates for the dsRNA molecules were produced based on publicly available or internally developed transcriptome information for the P. chrysocephala, P. nemorum, P. striolata, P. cruciferae and P. armoraciae targets. All the dsRNA samples tested were produced using primers designed automatically using Primer3, a primer design tool, to synthetize a dsRNA fragment of around 500-600 bp based on the coding sequence of each target gene. Smaller fragments were designed if the size of the coding sequence did not allow a 500 bp fragment.

### Example 2: Identification of Target Genes from Flea Beetle Species

The dsRNA molecules described above were tested for toxicity against their respective insect pest species in laboratory bioassays. In other words, dsRNAs based on coding sequences from P. chrysocephala were tested against P. chrysocephala insects, dsRNAs based on coding sequences from P. nemorum were tested against P. nemorum insects, dsRNAs based on coding sequences from P. striolata were tested against P. striolata insects, dsRNAs based on coding sequences from P. armoraciae were tested against P. armoraciae insects, and dsRNAs based on coding sequences from P. cruciferae were tested against P. cruciferae insects.

Identification of Target Genes from P. chrysocephala

Bioassays were performed using an RNA-treated artificial diet method. Briefly, synthesized dsRNA molecules were diluted to the appropriate concentration in a sucrose solution. Samples containing dsRNA and sucrose are heated up to 60° C. An agarose solution was heated to boiling and added to the dsRNA dilutions, leading to final concentrations of 5% sucrose and 0.5% agarose. The agarose solution containing the dsRNA was divided over three petri dishes (diameter=3 cm), the final dose being 67 μg of dsRNA per petri dish. Ten to twelve adults were added to each petri dish to have between 30 and 36 adults per treatment. Each petri dish was maintained at approximately 25° C. and 16:8 light:dark photoperiod. Mortality was recorded at different days post-infestation, with the final survival percentage calculated at 12 days with Abbott correction. dsRNA designed to target green fluorescent protein (GFP) was used as a negative control. Results are depicted in Table 1.

Identification of Target Genes from P. nemorum

Bioassays were performed using an RNA-treated artificial diet method. Briefly, synthesized dsRNA molecules were diluted to the appropriate concentration in a sucrose solution. Samples containing dsRNA and sucrose are heated up to 60° C. An agarose solution was heated to boiling and added to the dsRNA dilutions, leading to final concentrations of 5% sucrose and 0.5% agarose. The agarose solution containing the dsRNA was divided over three petri dishes (diameter=3 cm), the final dose being 67 μg of dsRNA per petri dish. Ten to twelve adults were added to each petri dish to have between 30 and 36 adults per treatment. Each petri dish was maintained at approximately 25° C. and 16:8 light:dark photoperiod. Mortality was recorded at different days post-infestation, with the final survival percentage calculated at 12 days with Abbott correction. dsRNA designed to target green fluorescent protein (GFP) was used as a negative control. Results are depicted in Table 1.

Identification of Target Genes from P. striolata

Bioassays were performed using an RNA-treated leaf disc method. Briefly, synthesized dsRNA molecules were diluted to the appropriate concentration and applied to canola leaf discs (5 mm diameter), coating the top surface with a final dose of 20 ng/mm². Leaf disks were placed in 20 mL plastic cups with ten beetles per cup. Beetles were fed fresh leaf discs, coated with dsRNA, every second day, for a period of two weeks. The cups were maintained at approximately 25° C. and 16:8 light:dark photoperiod. The number of dead adults was recorded every second day (when fresh leaf discs are provided), with the final survival percentage calculated at 14 days with Abbott correction. dsRNA designed to target green fluorescent protein (GFP) was used as a negative control. Results are depicted in Table 1.

Identification of Target Genes from P. armoraciae

Bioassays are performed using an RNA-treated artificial diet method. Briefly, synthesized dsRNA molecules are diluted to the appropriate concentration in a sucrose solution. The solution containing the dsRNA is divided over three wells of a 96-well plate, the final dose being 1 μg/μl dsRNA in 5% sucrose per well. The plates are then sealed and six pinholes per well are made through the seals. Eight adults per well are added to a 6-well plate which is attached to the reversed sample plate by the use of tape, allowing the insects to feed on the droplets. On day 3 the dsRNA solution is replaced by a 15% sucrose solution to keep the insects until the end of the assay. Each assay is maintained at approximately 25° C. and 16:8 light:dark photoperiod. Mortality is recorded at different days post-infestation, with the final survival percentage calculated at 10 up to 17 days with Abbott correction. dsRNA designed to target green fluorescent protein (GFP) is used as a negative control. dsRNA designed to the specific ubiquitin protein is used as a positive control. Results are depicted in Table 1.

Identification of Target Genes from P. cruciferae

Bioassays are performed using an RNA-treated artificial diet method. Briefly, synthesized dsRNA molecules are diluted to the appropriate concentration in a sucrose solution. The solution containing the dsRNA is divided over three wells of a 96-well plate, the final dose being 1 μg/μl dsRNA in 5% sucrose per well. The plates are then sealed and six pinholes per well are made through the seals. Eight adults per well are added to a 6-well plate which is attached to the reversed sample plate by the use of tape, allowing the insects to feed on the droplets. On day 3 the dsRNA solution is replaced by a 15% sucrose solution to keep the insects until the end of the assay. Each assay is maintained at approximately 25° C. and 16:8 light:dark photoperiod. Mortality is recorded at different days post-infestation, with the final survival percentage calculated at 10 up to 17 days with Abbott correction. dsRNA designed to target green fluorescent protein (GFP) is used as a negative control. dsRNA designed to the specific ubiquitin protein is used as a positive control. Results are depicted in Table 1.

For Table 1, the target gene is named. The Target ID is also named for each ortholog from each flea beetle species tested. “Pc” indicates the target ID is from P. chrysocephala, “Pn” indicates the target ID is from P. nemorum, “Ps” indicates the target ID is from P. striolata, “Pa” indicates the target ID is from P. armoraciae, and “Pu” indicates the target ID is from P. cruciferae. The “SEQ ID NO.” indicates the RNA sequence of the sense strand of the dsRNA tested. “Survival %” is the final survival percentage calculated, with Abbott correction, for each insect bioassay.

TABLE 1
Activity of dsRNA molecules against flea beetle species

| | Pa | | | Pu | | |
|---|---|---|---|---|---|---|
| | Target | SEQ ID | Survival | Target | SEQ ID | Survival |
| Target gene | ID | NO. | % | ID | NO. | % |
| GFP | | | 100 | | | 100 |
| Rpn12 | Pa1 | 511 | 0 | Pu1 | 596 | 33.3 |
| RpS13 | Pa2 | 512 | 38.6 | Pu2 | 597 | |
| RpS3A | Pa3 | 513 | | Pu3 | 598 | |
| Prosbeta2 | Pa4 | 514 | | Pu4 | 599 | |
| RpL11 | Pa5 | 515 | | Pu5 | 600 | |
| RpS18 | Pa6 | 516 | | Pu6 | 601 | |
| alphaSnap | Pa7 | 517 | 32.0 | Pu7 | 602 | 23.3 |
| His2B | Pa8 | 518 | 36.8 | Pu8 | 603 | 60.7 |
| Vha13 | Pa9 | 519 | 17.5 | Pu9 | 604 | |
| His2A | Pa10 | 520 | | Pu10 | 605 | |
| RpII215 | Pa11 | 521 | 15.3 | Pu11 | 606 | 0 |
| RpL18A | Pa12 | 522 | | Pu12 | 607 | |
| RpL27 | Pa13 | 523 | | Pu13 | 608 | |
| RpL32 | Pa14 | 524 | | Pu14 | 609 | |
| Rpb4 | Pa15 | 525 | 9.1 | Pu15 | 610 | |
| Cpsf73 | Pa16 | 526 | 57.1 | Pu16 | 611 | |
| Sf3b1 | Pa17 | 527 | 43.9 | Pu17 | 612 | 10.1 |
| | Ps | | | Pc | | |
| | Target | SEQ ID | Survival | Target | SEQ ID | Survival |
| Target gene | ID | NO. | % | ID | NO | % |
| GFP | | | 100 | | | 100 |
| Rpn12 | Ps1 | 562 | 17.8 | Pc1 | 528 | 66.7 |
| RpS13 | Ps2 | 563 | 35.6 | Pc2 | 529 | |
| RpS3A | Ps3 | 564 | 0 | Pc3 | 530 | |
| Prosbeta2 | Ps4 | 565 | 6.7 | Pc4 | 531 | |
| RpL11 | Ps5 | 566 | 0 | Pc5 | 532 | |
| RpS18 | Ps6 | 567 | 8.9 | Pc6 | 533 | |
| alphaSnap | Ps7 | 568 | 8.9 | Pc7 | 534 | 24.3 |
| His2B | Ps8 | 569 | 17.8 | Pc8 | 535 | 33.3 |
| Vha13 | Ps9 | 570 | 17.8 | Pc9 | 536 | 16.7 |
| His2A | Ps10 | 571 | 26.7 | Pc10 | 537 | |
| RpII215 | Ps11 | 572 | | Pc11 | 538 | |
| RpL18A | Ps12 | 573 | 17.8 | Pc12 | 539 | |
| RpL27 | Ps13 | 574 | 0 | Pc13 | 540 | |
| RpL32 | Ps14 | 575 | 0 | Pc14 | 541 | |
| Rpb4 | Ps15 | 576 | 46.2 | Pc15 | 542 | |
| Cpsf73 | Ps16 | 577 | | Pc16 | 543 | |
| Sf3b1 | Ps17 | 578 | | Pc17 | 544 | |

Table 1a,b shows the insecticidal activity of the selected targets for each of the flea beetle insects analyzed. It has previously been suggested that certain genes of a given insect species can be predicted to confer an RNAi-mediated insecticidal effect based on the essential nature of the gene in insect of a different genus. However, empirical evaluation of the target genes revealed that the insecticidal effect could not be predicted (See Baum et al., 2007, Nature Biotechnology 25: 1322-1326; also U.S. Publication No. 2015/0322456). Additionally, it has been suggested that a gene which has been shown to be a useful target for RNAi-mediated insect control for one insect pest is a useful target for RNAi-mediated insect control of a second insect pest of a different genus and/or family. However, empirical evaluation of the target gene in different insect pests of different families show that a given target with very high insecticidal activity in one insect pest may not produce significant mortality or growth inhibition in a second insect pest (Knorr et al, 2018, Scientific Reports 8: 2061, DOI: 10.1038/s41598-018-20416-y). Therefore, the insecticidal activity of a dsRNA molecule against a target gene of an insect pest can only be determined empirically.

### Example 3: Dose Response Curves of Selected dsRNA Molecules Against Flea Beetle Species

This example describes testing dsRNA molecules of the invention for biological activity against flea beetle species in further laboratory bioassays. In other words, dsRNAs based on coding sequences from P. chrysocephala were further tested against P. chrysocephala insects, dsRNAs based on coding sequences from P. nemorum were further tested against P. nemorum insects, dsRNAs based on coding sequences from P. striolata were further tested against P. striolata insects, dsRNAs based on coding sequences from P. armoraciae were tested against P. armoraciae insects, and dsRNAs based on coding sequences from P. cruciferae were tested against P. cruciferae insects.

Dose Response Curves of Selected dsRNA Molecules Against P. chrysocephala

The dsRNA molecules described above are tested for toxicity against P. chrysocephala in laboratory bioassays in a dilution series including a 2-fold and 10-fold dilution to generate dose response curves (DRC). Bioassays are performed using an RNA-treated artificial diet method. Briefly, synthesized dsRNA molecules are diluted to the appropriate concentration in a sucrose solution. Samples containing dsRNA and sucrose are heated up to 60° C. An agarose solution is heated till boiling and added to the dsRNA dilutions, leading to final concentrations of 5% sucrose and 0.5% agarose. The agarose solution containing the dsRNA is divided over three petri dishes (diameter=3 cm), the final dose being 67 μg, 33 μg or 7 μg of dsRNA per petri dish. Ten to twelve adults are added to each petri dish to have between 30 and 36 adults per treatment. Each petri dish is maintained at approximately 25° C. and 16:8 light:dark photoperiod. Mortality is recorded at different days post-infestation, with percent survival (% survival) calculated on day 12 with an Abbott correction. dsRNA designed to target GFP is used as a negative control.

Dose Response Curves of Selected dsRNA Molecules Against P. nemorum

The dsRNA molecules described above are tested for toxicity against P. nemorum in laboratory bioassays in a dilution series including a 2-fold and 10-fold dilution to generate dose response curves (DRC). Bioassays are performed using an RNA-treated artificial diet method. Briefly, synthesized dsRNA molecules are diluted to the appropriate concentration in a sucrose solution. Samples containing dsRNA and sucrose are heated up to 60° C. An agarose solution is heated till boiling and added to the dsRNA dilutions, leading to final concentrations of 5% sucrose and 0.5% agarose. The agarose solution containing the dsRNA is divided over three petri dishes (diameter=3 cm), the final dose being 67 μg, 33 μg or 7 μg of dsRNA per petri dish. Ten to twelve adults are added to each petri dish to have between 30 and 36 adults per treatment. Each petri dish is maintained at approximately 25° C. and 16:8 light:dark photoperiod. Mortality is recorded at different days post-infestation, with percent survival (% survival) calculated on day 12 with an Abbott correction. dsRNA designed to target GFP is used as a negative control.

Dose Response Curves of Selected dsRNA Molecules Against P. striolata

The dsRNA molecules described above are tested for toxicity against P. striolata in laboratory bioassays in a dilution series including a 2-fold and 10-fold dilution to generate dose response curves (DRC). Bioassays are performed using an RNA-treated leaf disc method. Briefly, synthesized dsRNA molecules are diluted to the appropriate concentration and applied to canola leaf discs (5 mm diameter), coating the top surface with a final dose of 20, 10 or 2 ng/mm2. Leaf disks are placed in 20 mL plastic cups with ten beetles per cup. Beetles are fed fresh leaf discs, coated with dsRNA, every second day, for a period of two weeks. The cups are maintained at approximately 25° C. and 16:8 light:dark photoperiod. The number of dead adults is recorded every second day (when fresh leaf discs are provided), with the final survival percentage calculated at 14 days. dsRNA designed to target green fluorescent protein (GFP) is used as a negative control.

### Example 4: Testing of Other dsRNA Sub-Fragments of Selected Targets Against Flea Beetle Species

Testing of Other dsRNA Sub-Fragments of Selected Targets Against P. chrysocephala

This example describes testing other sub-fragments of dsRNA molecules of the invention for biological activity against P. chrysocephala. These sub-fragments are based on the coding sequence of a selection of positive targets, and are either a shorter length or are based on a different region of the coding sequence compared to the initial dsRNA fragment.

The dsRNA molecules described below are tested for toxicity against P. chrysocephala in laboratory bioassays. Bioassays are performed using an RNA-treated artificial diet method. Briefly, synthesized dsRNA molecules are diluted to the appropriate concentration in a sucrose solution. Samples containing dsRNA and sucrose are heated up to 60° C. An agarose solution is heated to boiling and added to the dsRNA dilutions, leading to final concentrations of 5% sucrose and 0.5% agarose. The agarose solution containing the dsRNA is divided over three petri dishes (diameter=3 cm), the final dose being 67 μg of dsRNA per petri dish. Ten to twelve adults are added to each petri dish to have between 30 and 36 adults per treatment. Each petri dish is maintained at approximately 25° C. and 16:8 light:dark photoperiod. Mortality is recorded at different days post-infestation, with the final survival percentage calculated at 12 days with Abbott correction. dsRNA designed to target green fluorescent protein (GFP) is used as a negative control.

Testing of Other dsRNA Sub-Fragments of Selected Targets Against P. nemorum

This example describes testing other sub-fragments of dsRNA molecules of the invention for biological activity against P. nemorum. These sub-fragments are based on the coding sequence of a selection of positive targets, and are either a shorter length or are based on a different region of the coding sequence compared to the initial dsRNA fragment.

The dsRNA molecules described below are tested for toxicity against P. nemorum in laboratory bioassays. Bioassays are performed using an RNA-treated artificial diet method. Briefly, synthesized dsRNA molecules are diluted to the appropriate concentration in a sucrose solution. Samples containing dsRNA and sucrose are heated up to 60° C. An agarose solution is heated to boiling and added to the dsRNA dilutions, leading to final concentrations of 5% sucrose and 0.5% agarose. The agarose solution containing the dsRNA is divided over three petri dishes (diameter=3 cm), the final dose being 67 μg of dsRNA per petri dish. Ten to twelve adults are added to each petri dish to have between 30 and 36 adults per treatment. Each petri dish is maintained at approximately 25° C. and 16:8 light:dark photoperiod. Mortality is recorded at different days post-infestation, with the final survival percentage calculated at 12 days with Abbott correction. dsRNA designed to target green fluorescent protein (GFP) is used as a negative control.

Testing of Other dsRNA Sub-Fragments of Selected Targets Against P. striolata

This example describes testing other sub-fragments of dsRNA molecules of the invention for biological activity against P. striolata. These sub-fragments are based on the coding sequence of a selection of positive targets, and are either a shorter length or are based on a different region of the coding sequence compared to the initial dsRNA fragment.

The dsRNA molecules described below are tested for toxicity against P. striolata in laboratory bioassays. Bioassays are performed using an RNA-treated leaf disc method. Briefly, synthesized dsRNA molecules are diluted to the appropriate concentration and applied to canola leaf discs (5 mm diameter), coating the top surface with a final dose of 20 ng/mm2. Leaf disks are placed in 20 mL plastic cups with ten beetles per cup. Beetles are fed fresh leaf discs, coated with dsRNA, every second day, for a period of two weeks. The cups are maintained at approximately 25° C. and 16:8 light:dark photoperiod. The number of dead adults is recorded every second day (when fresh leaf discs are provided), with the final survival percentage calculated at 14 days with Abbott correction. dsRNA designed to target green fluorescent protein (GFP) is used as a negative control.

### Example 5. Producing Targeted dsRNA Molecules by Bacterial Expression

This example describes producing dsRNA molecules engineered against identified flea beetle targets using a bacterial expression system.

A hairpin cassette is engineered for at least one selected flea beetle target (a P. chrysocephala, P. nemorum, P. striolata, P. armoraciae and/or P. crucifera target). The hairpin cassette comprises a T7 promoter operably linked to an antisense sequence of the target, further linked at the 3′end to a nucleic acid sequence which is capable of forming a loop structure, further linked at the 3′end to the corresponding sense sequence of the target, operably linked at the 3′end to a T7 terminator sequence. The hairpin cassette is introduced into bacterial expression vector pGCP295 via BamHI and NotI restriction sites. The vector is then introduced into Escherichia coli strain HT115(DE3)GA01 via electroporation using standard methods, and transformants are selected for using kanamycin selection.

The bacteria containing the targeted dsRNA expression vector plasmid are grown in defined medium to a specific optical density and induced by addition of IPTG for a specific time period following standard methods and routine optimization. After induction, the bacteria are harvested by centrifugation, and the produced dsRNA molecules are collected.

### Example 6: Activity of dsRNA Molecules in a Spray Application Assay

This example describes testing of a sub-set of the identified target dsRNAs of the invention for biological activity against flea beetles when applied as a spray. The target dsRNAs may be derived from target genes from P. chrysocephala, P. nemorum, P. striolata, P. armoraciae and/or P. cruciferae. dsRNA molecules are bacterially produced as described above for testing as a spray application on planta. A negative control GFP dsRNA molecule is also produced.

Three 2-3 week old oil seed rape plants (Brassica napus L.) are sprayed with bacterially produced dsRNA (for example, 200 μg per 3 plants), dissolved in a sucrose solution. 15 insects are put on the plant after spraying, and the infested plant is maintained in a container closed with a mesh. Three days after the initial spraying, the plant is replaced by a second plant sprayed with the same bacterial lysate sucrose solution. Six days after the initial spraying the insects are transferred to an untreated plant. The number of dead adults is recorded every two to three days.

### Example 7: Expression of an Interfering RNA Molecule Comprising Target dsRNA in Canola Plants

This example describes introducing a construct that expresses an interfering RNA molecule into plant cells.

### Vector Construction

Expression vectors designed to produce hairpin RNAs (hpRNA) consist of a cassette containing a promoter, a sense strand, an intron functioning as a loop sequence, an antisense strand, and terminator. The hpRNA targets at least 21 nucleotides of an endogeneous gene target as described in Table 1. The hpRNA expression cassette is cloned into a binary vector. The binary vector also contains a second expression cassette between the left and right T-DNA borders which designed to express a selectable marker for selection of transgenic cells, tissues, and/or plants following plant transformation. The binary vector also contains selectable markers for selection of the presence of the binary vector bacteria.

Agrobacterium-Mediated Transformation of Brassica napus

Each resulting plasmid containing the hairpin cassette was transformed into Agrobacterium tumefaciens using standard molecular biology techniques known to those skilled in the art. The vectors described above were transformed into canola.

Stably transformed Brassica napus cv. ‘Westar’ events are obtained using an adapted published floral dip protocol (Wang et al. 2003). Adult flowering plants are infiltrated twice under vacuum with an Agrobacterium tumefaciens suspension. The strain used is C58C1RifR harbouring the pGV3101 Ti plasmid and a binary vector containing two plant expression cassettes, one for the insect target derived dsRNA which was inserted as a hairpin and an nptII based plant selectable marker.

After the two infiltrations, performed a week apart, the plants are allowed to mature and set seed. To identify the transformation events, the seed is soaked for 2 days in 300 mg/I kanamycin sulphate solution and then sown out into soil (Li et al. 2010). One week after sowing, the putative positive seedlings are identified due to their healthy green expanded cotyledons versus the smaller, shrunken bleached non-transformed seedlings.

The transgenic status of the putative events is checked by plus/minus PCR for the presence of the nptII sequence. Positive events are grown up to flowering at which point racemes are removed for insect bioassay.

### Transgenic Canola Feeding Assay

For the insect feeding bioassay, racemes are removed from the plant when in bud and immediately the cut end is put through the plastic lid of a 20 ml glass vial containing water. Each raceme is infested with 10-15 wild caught P. chrysocephala, P. nemorum, and/or P. striolata adults, and mortality is scored up till day 14 post infestation. The racemes are changed for fresh at day 7.

### Example 8: Interfering RNA Molecules with a Second Insecticidal Agent Bioassays

Double stranded RNA molecules are produced against a selected target. Additionally, a second insecticidal agent is prepared. Both the RNA and the second insecticidal agent are tested in combination for toxicity against P. chrysocephala, P. nemorum, and/or P. striolata in laboratory bioassays.

It should be understood that the examples and embodiments described herein are for illustrative purposes only and that various modifications or changes in light thereof of the description will be suggested to persons skilled in the art and are to be included within the spirit and purview of this application and the scope of the appended claims.

All publications and patent applications mentioned in this specification are indicative of the level of skill of those skilled in the art that this invention pertains.

Additional Particular Embodiments:

The following are particular embodiments of the invention:

1. An interfering ribonucleic acid (RNA) molecule wherein the RNA comprises at least one dsRNA wherein the dsRNA is a region of double-stranded RNA comprising annealed complementary strands, one strand of which comprises a sequence of at least 19 contiguous nucleotides which is at least partially complementary to a target nucleotide sequence within a flea beetle target gene, and (i) is at least 85% identical to at least a 19 contiguous nucleotide fragment of SEQ ID NO: 409-612, or the complement thereof; (ii) comprises at least a 19 contiguous nucleotide fragment of SEQ ID NO: 409-612, or the complement thereof; (iii) comprises at least a 19 contiguous nucleotide fragment of a nucleotide sequence encoding an amino acid sequence encoded by SEQ ID NO: 409-612, or the complement thereof, or (iv) can hybridize under stringent conditions to a polynucleotide comprising the nucleotide sequence of SEQ ID NO: 409-612, or the complements thereof, wherein the interfering RNA molecule has insecticidal activity on an insect pest, wherein the insect pest is a flea beetle of the Alticini tribe.
2. An interfering RNA molecule of claim 1, wherein said insect pest is a flea beetle is a species of the Phyllotreta or Psylliodes genera.
3. An interfering RNA molecule of claim 1 wherein the RNA comprises at least two dsRNAs, wherein each dsRNA comprises a sequence of nucleotides which is at least partially complementary to a target nucleotide sequence within the target gene.
4. An interfering RNA molecule of claim 3 wherein each of the dsRNAs comprise a different sequence of nucleotides which is at least partially complementary to a different target nucleotide sequence within the target gene.
5. The interfering RNA molecule of claim 1, wherein the interfering RNA molecule comprises SEQ ID NO: 409-612, or the complement thereof.
6. An interfering RNA molecule of claim 1, wherein the dsRNA is a region of double-stranded RNA comprising substantially complementary annealed strands.
7. An interfering RNA molecule of claim 1, wherein the dsRNA is a region of double-stranded RNA comprising fully complementary annealed strands.
8. An interfering RNA molecule of any one of claims 1 to 7, wherein the insect pest is a flea beetle selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).
9. A nucleic acid construct comprising the interfering RNA molecule of any of claims 1 to 8.
10. A nucleic acid molecule encoding the interfering RNA molecule of any of claims 1 to 8.
11. A nucleic acid construct comprising a nucleotide sequence that encodes the nucleic acid molecule of claim 10.
12. The nucleic acid construct of any of claim 9 or 11 wherein the nucleic acid construct is an expression vector.
13. A recombinant vector comprising a regulatory sequence operably linked to a nucleotide sequence that encodes the interfering RNA molecule of any one of claims 1 to 8.
14. A composition comprising two or more of the interfering RNA molecules of any of claims 1 to 8.
15. A composition of claim 14 wherein the two or more interfering RNA molecules are present on the same nucleic acid construct, on different nucleic acid constructs, or any combination thereof.
16. A composition of any of claim 14 or 15, comprising an interfering RNA molecule which comprises at least one dsRNA wherein the dsRNA is a region of double-stranded RNA comprising annealed complementary strands, one strand of which comprises a sequence of at least 19 contiguous nucleotides which (i) is at least 85% identical to at least a 19 contiguous nucleotide fragment of SEQ ID NO: 409-612, or complement thereof; or (ii) comprises at least a 19 contiguous nucleotide fragment of SEQ ID NO: 409-612, or complement thereof.
17. A composition comprising two or more of the nucleic acid constructs of any of claims 9, 11, or 12, wherein the two or more nucleic acid constructs each comprise a different interfering RNA.
18. A composition comprising two or more of the nucleic acid molecules of claim 10, wherein the two or more nucleic acid molecules each encode a different interfering RNA molecule.
19. An insecticidal composition for inhibiting the expression of a flea beetle target gene, comprising the interfering RNA of any one of claims 1 to 8 and an agriculturally acceptable carrier.
20. An insecticidal composition of claim 19 comprising at least a second insecticidal agent for controlling a flea beetle.
21. An insecticidal composition of claim 20 wherein the second insecticidal agent is a Bacillus thuringiensis insecticidal protein.
22. An insecticidal composition of claim 20 wherein the second insecticidal agent is not a Bacillus thuringiensis insecticidal protein.
23. An insecticidal composition of claim 20 wherein the second insecticidal agent is a patatin, a protease, a protease inhibitor, a urease, an alpha-amylase inhibitor, a pore-forming protein, a lectin, an engineered antibody or antibody fragment, or a chitinase.
24. An insecticidal composition of claim 22 wherein the second insecticidal agent is or is derived from a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, or a Clostridium spp. insecticidal protein.
25. A transgenic plant, or part thereof, comprising the interfering RNA molecule, the nucleic acid molecule, the nucleic acid construct, and/or the composition of any of the respective preceding claims, wherein the transgenic plant has enhanced resistance to a flea beetle as compared to a control plant.
26. A transgenic plant, or part thereof, of claim 25, wherein the transgenic plant comprises at least a second insecticidal agent for controlling flea beetles.
27. A transgenic plant, or part thereof, of claim 26, wherein the second insecticidal agent is a Bacillus thuringiensis insecticidal protein.
28. A transgenic plant, or part thereof, of claim 26, wherein the second insecticidal agent is not a Bacillus thuringiensis insecticidal protein.
29. A transgenic plant, or part thereof, of claim 26, wherein the second insecticidal agent is a patatin, a protease, a protease inhibitor, a urease, an alpha-amylase inhibitor, a pore-forming protein, a lectin, an engineered antibody or antibody fragment, or a chitinase.
30. The transgenic plant, or part thereof, of claim 28, wherein the second insecticidal agent is or is derived from a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, or a Clostridium spp. insecticidal protein.
31. A transgenic plant, or part thereof, of any one of claims 25 to 30, wherein the transgenic plant, or part thereof, is a canola plant or part thereof.
32. Transgenic seed of a transgenic plant of any one of claims 25 to 31.
33. A biological sample from the transgenic plant, or part thereof, of any one of claims 25 to 31.
34. A commodity product derived from the transgenic plant, or part thereof, of any one of claims 25 to 31.
35. A commodity product of claim 34, wherein the commodity product is selected from the group consisting of whole or processed seeds, kernels, hulls, meals, grits, flours, sugars, sugars, starches, protein concentrates, protein isolates, waxes, oils, extracts, juices, concentrates, liquids, syrups, feed, silage, fiber, paper or other food or product produced from plants.
36. A method of controlling a flea beetle comprising contacting the flea beetle with a nucleic acid molecule that is or is capable of producing an interfering RNA molecule of claims 1-8 for inhibiting expression of a target gene in the flea beetle thereby controlling the flea beetle.
37. The method of claim 36, wherein the target gene comprises a coding sequence which:

a) is at least 85% identical to at least a 19 nucleotide contiguous fragment of SEQ ID NO: 1-102, or a complement thereof;

b) comprises at least a 19 nucleotide contiguous fragment of SEQ ID NO: 1-102, or a complement thereof; or

c) comprises at least a 19 nucleotide contiguous fragment of a nucleotide sequence encoding an amino acid sequence encoded by SEQ ID NO: 1-102, or a complement thereof.

38. The method of claim 36 wherein the interfering RNA molecule comprises at least one dsRNA, wherein the dsRNA is a region of double-stranded RNA comprising annealed complementary strands, one strand of which comprises a sequence of at least 19 contiguous nucleotides which (i) is at least 85% identical to at least a 19 contiguous nucleotide fragment of SEQ ID NO: 409-612, or the complement thereof; or (ii) comprises at least a 19 contiguous nucleotide fragment of SEQ ID NO: 409-612, or the complement thereof; or (iii) comprises at least a 19 contiguous nucleotide fragment of a nucleotide sequence encoding an amino acid sequence encoded by SEQ ID NO: 409-612, or the complement thereof.
39. The method of any one of claims 36 to 38, wherein the flea beetle is selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).
40. The method of claim 39, wherein contacting comprises:

a) planting a transgenic seed capable of producing a transgenic plant that expresses the nucleic acid molecule, wherein the flea beetle feeds on the transgenic plant, or part thereof; or

b) applying a composition comprising the nucleic acid molecule to a seed or plant, or part thereof, wherein the flea beetle feeds on the seed, the plant, or a part thereof.

41. The method of claim 39, wherein the transgenic seed and transgenic plant is a canola seed and a canola plant.
42. The method of claim 39, wherein the seed or plant is a canola seed or canola plant.
43. A method of controlling a flea beetle comprising contacting the flea beetle with a nucleic acid molecule that is or is capable of producing the interfering RNA molecule of claims 1-8 for inhibiting expression of a target gene in the flea beetle, and contacting the flea beetle with at least a second insecticidal agent for controlling the flea beetle.
44. A method of controlling a flea beetle comprising contacting the flea beetle with a nucleic acid molecule that is or is capable of producing the interfering RNA molecule of claims 1-8 for inhibiting expression of a target gene in the flea beetle, and contacting the flea beetle with at least a second insecticidal agent for controlling the flea beetle, thereby controlling the flea beetle, wherein said second insecticidal agent comprises a B. thuringiensis insecticidal protein.
45. A method of controlling a flea beetle comprising contacting the flea beetle with a nucleic acid molecule that is or is capable of producing the interfering RNA molecule of claims 1-8 for inhibiting expression of a target gene in the flea beetle, and contacting the flea beetle with at least a second insecticidal agent for controlling the flea beetle, thereby controlling the flea beetle, wherein said second insecticidal agent does not comprise a B. thuringiensis insecticidal protein.
46. The method of claim 43, wherein the second insecticidal agent comprises a patatin, a protease, a protease inhibitor, a urease, an alpha-amylase inhibitor, a pore-forming protein, a lectin, an engineered antibody or antibody fragment, or a chitinase.
47. The method of claim 45, wherein the second insecticidal agent comprises a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, or a Clostridium spp. insecticidal protein.
48. A method of reducing an adult flea beetle population on a transgenic plant expressing a Cry protein, a hybrid Cry protein or modified Cry protein comprising expressing in the transgenic plant a nucleic acid molecule that is or is capable of producing an interfering RNA molecule of claim 1, which inhibits expression of a target gene in an adult flea beetle thereby reducing the adult flea beetle population.
49. A method of reducing resistance development in a flea beetle population to an interfering RNA molecule of claim 1, the method comprising expressing in a transgenic plant fed upon by the flea beetle population an interfering RNA molecule of claim 1 which inhibits expression of a target gene in a larval and adult flea beetle, thereby reducing resistance development in the flea beetle population compared to a flea beetle population exposed to an interfering RNA molecule capable of inhibiting expression of a target gene in a larval or adult flea beetle.
50. A method of reducing the level of a target RNA transcribed from a target gene in a flea beetle comprising contacting the flea beetle with a composition comprising the interfering RNA molecule of any one of claims 1 to 8, wherein the interfering RNA molecule reduces the level of the target RNA in a cell of the flea beetle.
51. The method of claim 50, wherein production of the protein encoded by the target RNA is reduced.
52. The method of claim 51, wherein the protein comprises an amino acid sequence encoded by a nucleic acid sequence with at least 85% identity to SEQ ID NO: 1-102 or the complement thereof.
53. The method of claim 50, wherein the interfering RNA is from a transgenic organism expressing the interfering RNA.
54. The method of claim 53, wherein the transgenic organism is a transgenic plant, a transgenic microorganism, a transgenic bacterium, or a transgenic endophyte.
55. The method of claim 50, wherein the interfering RNA is lethal to a flea beetle.
56. The method of claim 55, wherein the flea beetle is selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).
57. A method of conferring flea beetle tolerance to a plant, or part thereof, comprising introducing into the plant, or part thereof, the interfering RNA molecule, the nucleic acid molecule, the nucleic acid construct, and/or the composition of any of the respective preceding claims, thereby conferring tolerance of the plant or part thereof to the flea beetle.
58. A method of reducing damage to a plant fed upon by a flea beetle, comprising introducing into cells of the plant the interfering RNA molecule, the nucleic acid molecule, the nucleic acid construct, and/or the composition of any of the respective preceding claims, thereby reducing damage to the plant.
59. A method of producing a transgenic plant cell having toxicity to a flea beetle, comprising introducing into a plant cell the interfering RNA, the nucleic acid molecule, the nucleic acid construct, and/or the composition of any of the respective preceding claims, thereby producing the transgenic plant cell having toxicity to the flea beetle compared to a control plant cell.
60. A plurality of transgenic plant cells produced by the method of claim 59.
61. A plurality of transgenic plant cells of claim 60, wherein the plant cells are grown under conditions which include natural sunlight.
62. A method of producing a transgenic plant having enhanced tolerance to flea beetle feeding damage, comprising introducing into a plant the interfering RNA molecule, the nucleic acid molecule, or the nucleic acid construct of any of the respective preceding claims, thereby producing a transgenic plant having enhanced tolerance to flea beetle feeding damage compared to a control plant.
63. The method of claim 62, wherein the introducing step is performed by transforming a plant cell and producing the transgenic plant from the transformed plant cell.
64. The method of claim 62, wherein the introducing step is performed by breeding two plants together.
65. A method of enhancing control of a flea beetle population comprising providing the transgenic plant or plant part of claim 25 and applying to the plant or plant part a chemical pesticide. 66. The method of claim 65, wherein the chemical pesticide is a carbamate, a pyrethroid, an organophosphate, a friprole, a neonicotinoid, an organochloride, a nereistoxin, or a combination thereof. 67. The method of claim 65, wherein the chemical pesticide comprises an active ingredient selected from the group consisting of carbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof.
68. The method of claim 67, wherein the active ingredient is delivered in a product selected from the group consisting of Furadan®, Lanate®, Sevin®, Talstar®, Force®, Ammo®, Cymbush®, Delta Gold®, Karate®, Ambush®, Pounce®, Brigade®, Capture®, ProShield®, Warrior®, Dursban®, Fortress®, Mocap®, Thimet®, AAstar®, Rampart®, Counter®, Cygon®, Dicap®, Regent®, Cruiser®, Gaucho®, Prescribe®, Poncho®, Aztec®, and a combination thereof.
69. A method of providing a canola grower with a means of controlling a flea beetle pest population in a canola crop comprising (a) selling or providing to the grower transgenic canola seed that comprises an interfering RNA molecule, a nucleic acid molecule, a nucleic acid construct, and/or a composition of the invention; and (b) advertising to the grower that the transgenic canola seed produce transgenic canola plants that control a flea beetle pest population.
70. A method of identifying an orthologous target gene for using as a RNAi strategy for the control of a plant pest, said method comprising the steps of:
a) producing a primer pair that will amplify a target selected from the group comprising or consisting of SEQ ID NO: 1-102, or a complement thereof,
b) amplifying an orthologous target gene from a nucleic acid sample of the plant pest using the primer pair of step a),
c) identifying the sequence of the orthologous target gene amplified in step b),
d) producing an interfering RNA molecule, wherein the RNA comprises at least one dsRNA, wherein the dsRNA is a region of double-stranded RNA comprising annealed complementary strands, one strand of which comprises a sequence of at least 19 contiguous nucleotides which is at least partially complementary to the orthologous target nucleotide sequence within the target gene, and
e) determining if the interfering RNA molecule of step d) has insecticidal activity on the plant pest;
   wherein if the interfering RNA has insecticidal activity on the plant pest target gene, an orthologous target gene for using in the control of a plant pest has been identified.

1. An interfering ribonucleic acid (RNA) molecule wherein the RNA comprises at least one dsRNA wherein the dsRNA is a region of double-stranded RNA comprising annealed complementary strands, one strand of which comprises a sequence of at least 19 contiguous nucleotides which is at least partially complementary to a target nucleotide sequence within a flea beetle target gene, and (i) is at least 85% identical to at least a 19 contiguous nucleotide fragment of SEQ ID NO: 409-612, or the complement thereof, (ii) comprises at least a 19 contiguous nucleotide fragment of SEQ ID NO: 409-612, or the complement thereof, (iii) comprises at least a 19 contiguous nucleotide fragment of a nucleotide sequence encoding an amino acid sequence encoded by SEQ ID NO: 409-612, or the complement thereof, or (iv) can hybridize under stringent conditions to a polynucleotide comprising the nucleotide sequence of SEQ ID NO: 409-612, or the complements thereof, wherein the interfering RNA molecule has insecticidal activity on an insect pest, wherein the insect pest is a flea beetle of the Alticini tribe.

2. The interfering RNA molecule of claim 1, wherein the interfering RNA molecule comprises SEQ ID NO: 409-612, or the complement thereof.

3. An interfering RNA molecule of claim 1, wherein the dsRNA is a region of double-stranded RNA comprising substantially complementary annealed strands.

4. An interfering RNA molecule of claim 1, wherein the dsRNA is a region of double-stranded RNA comprising fully complementary annealed strands.

5. An interfering RNA molecule of claim 1, wherein the insect pest is a flea beetle selected from the group consisting of Phyllotreta armoraciae (horseradish flea beetle), Phyllotreta cruciferae (canola flea beetle), Phyllotreta pusilla (western black flea beetle), Phyllotreta nemorum (striped turnip flea beetle), Phyllotreta atra (turnip flea beetle), Phyllotreta robusta (garden flea beetle), Phyllotreta striolata (striped flea beetle), Phyllotreta undulata, Psylliodes chrysocephala, and Psylliodes punctulata (hop flea beetle).

6. A nucleic acid construct comprising the interfering RNA molecule of claim 1.

7. A recombinant vector comprising a regulatory sequence operably linked to a nucleotide sequence that encodes the interfering RNA molecule of claim 1.

8. A composition comprising two or more of the interfering RNA molecules of claim 1.

9. An insecticidal composition for inhibiting the expression of a flea beetle target gene, comprising the interfering RNA of claim 1 and an agriculturally acceptable carrier.

10. An insecticidal composition of claim 9 comprising at least a second insecticidal agent for controlling a flea beetle.

11. An insecticidal composition of claim 10 wherein the second insecticidal agent is or is derived from a Bacillus cereus insecticidal protein, a Xenorhabdus spp. insecticidal protein, a Photorhabdus spp. insecticidal protein, a Brevibacillus laterosporous insecticidal protein, a Lysinibacillus sphearicus insecticidal protein, a Chromobacterium spp. insecticidal protein, a Yersinia entomophaga insecticidal protein, a Paenibacillus popiliae insecticidal protein, or a Clostridium spp. insecticidal protein.

12. A transgenic plant, or part thereof, comprising the interfering RNA molecule, the nucleic acid molecule, the nucleic acid construct, and/or the composition of any of the respective preceding claims, wherein the transgenic plant has enhanced resistance to a flea beetle as compared to a control plant.

13. Transgenic seed of a transgenic plant of claim 12.

14. (canceled)

15. (canceled)

16. A method of controlling a flea beetle comprising contacting the flea beetle with a nucleic acid molecule that is or is capable of producing an interfering RNA molecule of claim 1 for inhibiting expression of a target gene in the flea beetle thereby controlling the flea beetle.

17. A method of controlling a flea beetle comprising contacting the flea beetle with a nucleic acid molecule that is or is capable of producing the interfering RNA molecule of claim 1 for inhibiting expression of a target gene in the flea beetle, and contacting the flea beetle with at least a second insecticidal agent for controlling the flea beetle, thereby controlling the flea beetle, wherein said second insecticidal agent comprises a B. thuringiensis insecticidal protein.
